(19) United States
(12) Patent Application Publication
(10) Pub. No.: US 2022/0143222 A1
(43) Pub. Date: May 12, 2022

(54) SUBCUTANEOUS AND INTRAMUSCULAR ADMINISTRATION OF PYRAZINE COMPOUNDS

(71) Applicant: MediBeacon Inc., St. Louis, MO (US)

(72) Inventors: Thomas E. Rogers, Ballwin, MO (US); Steven J. Hanley, St. Louis, MO (US); Richard B. Dorshow, St. Louis, MO (US)

(21) Appl. No.: 17/523,659

(22) Filed: Nov. 10, 2021

Related U.S. Application Data
(60) Provisional application No. 63/111,962, filed on Nov. 10, 2020.

(51) Int. Cl. A61K 49/00

(52) U.S. Cl. CPC A61K 49/0021; A61K 49/0004; A61K 49/0052

(57) ABSTRACT

The present disclosure relates to methods for determining the renal glomerular filtration rate or assessing the renal function in a patient in need thereof. The method comprises administering, subcutaneously or intramuscularly, a pyrazine compound of Formula I to a patient, wherein the administration produces a plasma concentration of the compound that is substantially similar to a plasma concentration produced by intravenous administration of an identical amount of the compound; and monitoring the rate in which the kidneys of the patient eliminate the pyrazine from the systemic circulation of the patient. The pyrazine compound fluoresces when exposed to electromagnetic radiation which may be detected using one or more sensors. The rate in which the fluorescence decreases in the patient may be used to calculate the renal glomerular filtration rate in the patient.

### CROSS-REFERENCE TO RELATED APPLICATION

This application claims benefit of U.S. Provisional Patent Application No. 63/111,962 which was filed in the United States Patent and Trademark Office on Nov. 10, 2020, the entire contents of which are incorporated herein by reference for all purposes.

### FIELD

The field of the disclosure generally relates to pharmaceutical compositions comprising pyrazine compounds and methods to transdermally detect fluorescence therefrom after subcutaneous or intramuscular administration, as well as use of said pharmaceutical compositions to assess renal function of a patient in need thereof.

### BACKGROUND

Acute renal failure (ARF) is a common ailment in patients admitted to general medical-surgical hospitals. Approximately half of the patients who develop ARF die either directly from ARF or from complications associated with an underlying medical condition, while survivors face marked increases in morbidity and prolonged hospitalization. Early diagnosis is generally believed to be important because renal failure is often asymptomatic and typically requires careful tracking of renal function markers in the blood. Dynamic monitoring of renal functions of patients is desirable in order to minimize the risk of acute renal failure brought about by various clinical, physiological and pathological conditions. Such dynamic monitoring tends to be particularly important in the case of critically ill or injured patients because a large percentage of these patients tend to face risk of multiple organ failure (MOF) potentially resulting in death. MOF is a sequential failure of the lungs, liver and kidneys and is incited by one or more of acute lung injury (ALI), adult respiratory distress syndrome (ARDS), hypermetabolism, hypotension, persistent inflammatory focus and sepsis syndrome. The common histological features of hypotension and shock leading to MOF generally include tissue necrosis, vascular congestion, interstitial and cellular edema, hemorrhage and microthrombi. These changes generally affect the lungs, liver, kidneys, intestine, adrenal glands, brain and pancreas in descending order of frequency. The transition from early stages of trauma to clinical MOF generally corresponds with a particular degree of liver and renal failure as well as a change in mortality risk from about 30% up to about 50%.

Traditionally, renal function of a patient has been determined using crude measurements of the patient's urine output and plasma creatinine levels. These values are frequently misleading because such values are affected by age, state of hydration, renal perfusion, muscle mass, dietary intake, and many other clinical and anthropometric variables. In addition, a single value obtained several hours after sampling may be difficult to correlate with other physiologic events such as blood pressure, cardiac output, state of hydration and other specific clinical events (e.g., hemorrhage, bacteremia, ventilator settings and others).

Chronic Kidney Disease (CKD) is a medical condition characterized in the gradual loss of kidney function over time. It includes conditions that damage the kidneys and decrease their ability to properly remove waste products from the blood of an individual. Complications from CKD include high blood pressure, anemia (low blood count), weak bones, poor nutritional health and nerve damage in addition to an increased risk of heart disease. According to the National Kidney Foundation, approximately two-thirds of all cases of CKD are caused by diabetes or hypertension. In addition to a family history of kidney disease, other risk factors include age, ethnicity, hypertension, and diabetes. The renal glomerular filtration rate (GFR) is the best test to determine the level of kidney function and assess the stage of a patient's CKD.

GFR is an important test to determine the level of kidney function which determines the state of CKD. The lower the GFR, the more serious the CKD. GFR can be estimated based on a blood test measuring the blood creatinine level in combination with other factors. More accurate, and therefore more useful, methods require the injection of an substance into a patient followed by careful monitoring of urine output over a period of time. These are often contrast agents (CA) that can cause renal problems on their own. Radioisotopes or iodinated aromatic rings are two common categories of CAs that are used for GFR determination.

Pyrazine derivatives are known in the art for use in renal monitoring, including those disclosed in U.S. Pat. Nos. 8,155,000, 8,481,734, 8,628,751, 8,664,392, 8,697,033, 8,722,685, 8,628,751, 8,778,309, 9,005,581, 9,216,963, 9,283,288, 9,376,399, 10,0525,149, 10,617,687, U.S. RE47413, and U.S. RE47255. Pyrazine derivatives are typically dosed intravenously (IV) for accurate determination of renal glomerular filtration rate by transdermal fluorescence. However, it would be beneficial to be able to utilize other routes of administration.

### SUMMARY OF THE INVENTION

In an aspect, the present disclosure encompasses a method for determining a glomerular filtration rate (GFR) in a patient in need thereof. The method comprises subcutaneously or intramuscularly administering to said patient about 3 mg to about 300 mg of a compound of Formula I or a pharmaceutically acceptable salt thereof as a 60-300 mg/mL solution, wherein the administration produces a plasma concentration of the compound that is substantially similar to a plasma concentration produced by intravenous administration of an identical amount of the compound; measuring a concentration of the compound of Formula I in said patient over a measurement time window; and determining the GFR in said patient using the measured concentration of the compound, wherein Formula I is

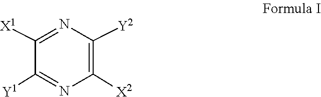

wherein each of X¹ and X² are independently chosen from —CO(AA), —CN, —CO₂R¹, —CONR²R³, —COR⁴, —NO₂, —SOR³⁵, —SO₂R⁶, —SO₂OR and —PO₃R⁸R⁹; each Y¹ and Y² are independently chosen from —OR¹⁰, —SR¹¹, —NR¹²R¹³, —N(R¹⁴)COR¹⁵, —CONH(PS); —P(R¹⁵)₂, —P(OR¹⁷)₂; and

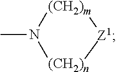

Z¹ is a single bond, —CR¹⁸R¹⁹—, —O—, —NR²⁰—, —NCOR²¹—, —S—, —O—, and —SO₂—; each R¹ to R²¹ are independently chosen from hydrogen, C₁-C₁₀ alkyl optionally substituted with hydroxyl and carboxylic acid, C₃-C₆ polyhydroxylated alkyl, C₅-C₁₀ aryl, C₅-C₁₀ heteroaryl, C₃-C₅ heterocycloalkyl optionally substituted with C(O), —(CH₂)ₐCO₂H optionally substituted with C₅-C₁₀ heteroaryl, (CH₂)ₐCONR³⁰R³¹, —(CH₂)ₐNHSO₃⁻, —(CH₂)ₐNHSO₃H, —(CH₂)ₐOH, —(CH₂)ₐOPO₃⁻, —(CH₂)ₐOPO₃H₂, —(CH₂)ₐOPO₃H⁻, —(CH₂)ₐOR²², —(CH₂)ₐOSO₃⁻, —(CH₂)ₐOSO₃H, —(CH₂)ₐPO₃⁼, —(CH₂)ₐPO₃H₂, —(CH₂)ₐPO₃H⁻, —(CH₂)ₐSO₃⁻, —(CH₂)ₐSO₃H, —(CH₂)_{d}CO(CH₂CH₂O)_{c}R²³, —(CH₂)_{d}(CH₂CH₂O)_{c}R²⁴, —(CHCO₂H)ₐCO₂H, —CH₂(CHNH₂)ₐCH₂NR²⁵R²⁶, —CH₂(CHOH)ₐCO₂H, —CH₂(CHOH)ₐR²⁷, —CH[(CH₂)_{b}NH₂]ₐCH₂OH, —CH[(CH₂)_{b}NH₂]ₐCO₂H, and —(CH₂)ₐNR²⁸R²⁹; each R²² to R³¹ are independently chosen from hydrogen, C₁-C₁₀alkyl, and C₁-C₅-dicarboxylic acid; R³⁵ is chosen from C₁-C₁₀ alkyl optionally substituted with hydroxyl and carboxylic acid, C₃-C₆ polyhydroxylated alkyl, C₅-C₁₀ aryl, C₅-C₁₀ heteroaryl, C₃-C₅ heterocycloalkyl optionally substituted with C(O), —(CH₂)ₐCO₂H optionally substituted with C₅-C₁₀ heteroaryl, —(CH₂)ₐCONR³⁰R³¹, —(CH₂)ₐNHSO₃⁻, —(CH₂)ₐNHSO₃H, —(CH₂)ₐOH, —(CH₂)ₐOPO₃⁼, —(CH₂)ₐOPO₃H₂, —(CH₂)ₐOPO₃H⁻, —(CH₂)ₐOR²², —(CH₂)ₐOSO₃⁻, —(CH₂)ₐOSO₃H, —(CH₂)ₐPO₃⁼, —(CH₂)ₐPO₃H₂, —(CH₂)ₐPO₃H⁻, —(CH₂)ₐSO₃⁻, —(CH₂)ₐSO₃H, —(CH₂)_{d}CO(CH₂CH₂O)_{c}R²³, —(CH₂)_{d}(CH₂CH₂O)_{c}R²⁴, —(CHCO₂H)ₐCO₂H, —CH₂(CHNH₂)ₐCH₂NR²⁵R²⁶, —CH₂(CHOH)ₐCO₂H, —CH₂(CHOH)ₐR²⁷, —CH[(CH₂)_{b}NH₂]ₐCH₂OH, —CH[(CH₂)_{b}NH₂]ₐCO₂H, and —(CH₂)ₐNR²⁸R²⁹; (AA) is a polypeptide chain comprising one or more natural or unnatural amino acids linked together by peptide bonds amide bonds and each instance of (AA) may be the same or different than each other instance; (PS) is a sulfated or non-sulfated polysaccharide chain comprising one or more monosaccharide units connected by glycosidic linkages; and each ‘a’, ‘b’, and ‘d’ are independently chosen from 0 to 10, ‘c’ is chosen from 1 to 100 and each of ‘m’ and ‘n’ independently is an integer from 1 to 3.

In another aspect, the present disclosure encompasses a method of assessing organ function in a patient. The method comprises subcutaneously or intramuscularly administering to said patient about 3 mg to about 300 mg of a fluorescent compound as a 60-300 mg/mL solution, wherein the administration produces a plasma concentration of the fluorescent compound that is substantially similar to a plasma concentration produced by intravenous administration of an identical amount of the fluorescent compound; exposing said fluorescent compound to electromagnetic radiation, thereby causing spectral energy to emanate from said fluorescent compound; detecting the spectral energy emanated from said fluorescent compound; and assessing organ function of the patient based on the detected spectral energy; wherein the fluorescent compound is a compound of Formula I or a pharmaceutically acceptable salt thereof, and wherein Formula I is described as above.

In another aspect, the present disclosure encompasses a method of assessing renal function in a patient. The method comprises subcutaneously or intramuscularly administering to said patient about 3 mg to about 300 mg of a fluorescent compound as a 60-300 mg/mL solution, wherein the administration produces a plasma concentration of the fluorescent compound that is substantially similar to a plasma concentration produced by intravenous administration of an identical amount of the fluorescent compound; exposing said fluorescent compound to electromagnetic radiation, thereby causing spectral energy to emanate from said fluorescent compound; detecting the spectral energy emanated from said fluorescent compound; and assessing renal function of the patient based on the detected spectral energy; wherein the fluorescent compound is a compound of Formula I or a pharmaceutically acceptable salt thereof, and wherein Formula I is described as above.

Other aspects and iterations of the invention are described more thoroughly below.

### BRIEF DESCRIPTION OF THE FIGURES

The application file contains at least one figure executed in color. Copies of this patent application publication with color photographs will be provided by the Office upon request and payment of the necessary fee.

FIG. 1 is a graph showing MB-102 plasma concentration (ng/ml) v. time after IV administration (triangle) and subcutaneous administration (square, circle, and diamond).

FIG. 2 is a graph illustrating plasma concentration (dashed line, right y-axis, ng/ml) and transdermal fluorescence (solid line, left y-axis) of MB-102 v. time (x-axis, hours) post IV injection.

FIG. 3 is a graph illustrating MB-102 plasma concentration (dashed line, right y-axis, ng/ml) and transdermal fluorescence (solid line, left y-axis) v. time (x-axis, hours) after multi-needle subcutaneous injection into an animal.

FIG. 4 is a graph illustrating MB-102 plasma concentration (dashed line, right y-axis, ng/ml) and transdermal fluorescence (solid line, left y-axis) v. time (x-axis, hours) subject after multi-needle subcutaneous injection into an animal.

FIG. 5 is a graph illustrating MB-102 plasma concentration (dashed line, right y-axis, ng/ml) and transdermal fluorescence (solid line, left y-axis) v. time (x-axis, hours) after single needle subcutaneous injection into an animal.

FIG. 6 is a graph illustrating MB-102 plasma concentration (dashed line, right y-axis, ng/ml) v. time (x-axis, hours) after single needle intramuscular injection into an animal.

FIG. 7 is a graph illustrating MB-102 plasma concentration (dashed line, right y-axis, ng/ml) and transdermal fluorescence (solid line, left y-axis) v. time (x-axis, hours) after single needle intramuscular injection into an animal.

FIG. 8 is a graph illustrating MB-102 plasma concentration (green x, right y-axis, ng/ml) and transdermal fluorescence (solid line, left y-axis) v. time (x-axis, hours) after single needle subcutaneous injection into an animal (1539).

### DETAILED DESCRIPTION

When introducing elements of the present disclosure or embodiments thereof, the articles “a,” “an,” “the,” and “said” are intended to mean that there are one or more of the elements. The terms “comprising,” including,” and “having” are intended to be inclusive and mean that there may be additional elements other than the listed elements.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

All references herein to “pyrazine”, “pyrazine derivative”, “pyrazine molecule”, “pyrazine compound” or “pyrazine analog” apply to all compounds of Formula I. Additionally each reference to “pyrazine”, “pyrazine derivative”, “pyrazine molecule”, “pyrazine compound” or “pyrazine analog” includes all pharmaceutically acceptable salts thereof unless specifically stated otherwise. Salt forms may be charged or uncharged, and may be protonated to form the appropriate cation or deprotonated to form the appropriate anion. All aspects and embodiments disclosed herein are applicable to compounds of Formula I, and specific examples are only illustrative and non-limiting to the scope of the disclosure.

The term “intramuscular administration” refers to administration of a composition into a muscle. The term “subcutaneous administration” refers to administration of a composition into a tissue layer between the skin (i.e., dermis) and the muscle. The term “intradermal administration” refers to administration of a composition into the dermis. Intramuscular administration, subcutaneous administration, and intradermal administration are therefore distinct routes of administration, targeting different sites of the body.

The term “MB-102” refers to the compound 3,6-diamino-2,5-bis{N-(1R)-1-carboxy-2-hydroxyethyl]carbamoyl}pyrazine or (2R,2′R)-2,2′-((3,6-diaminopyrazine-2,5-dicarbonyl)bis(azanediyl))bis (3-hydroxy-propanoic acid).

The term “MB-404” refers to the compound N2,N5-bis(2,3-dihydroxypropyl)-3,6-bis[(S)-2,3-dihydroxypropylamino]pyrazine-2,5-dicarboxamide.

### I. Compounds of Formula I

In one aspect, disclosed herein is a pyrazine compound of Formula I, or a pharmaceutically acceptable salt thereof,

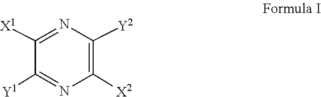

wherein each of X¹ and X² are independently chosen from —CO(AA), —CN, —CO₂R¹, —CONR²R³, —COR⁴, —NO₂, —SOR³⁵, —SO₂R⁶, —SO₂OR⁷ and —PO₃R⁸R⁹;
each Y¹ and Y² are independently chosen from —OR¹⁰, —SR¹¹, —NR¹²R¹³, —N(R¹⁴)COR¹⁵, —CONH(PS); —P(R¹⁶)₂, —P(OR¹⁷)₂ and

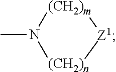

Z¹ is a single bond, —CR¹⁸R¹⁹—, —O—, —NR²⁰—, —NCOR²¹—, —S—, —SO—, and —SO₂—;
each R¹ to R²¹ are independently chosen from hydrogen, C₁-C₁₀ alkyl optionally substituted with hydroxyl and carboxylic acid, C₃-C₆ polyhydroxylated alkyl, C₅-C₁₀ aryl, C₅-C₁₀ heteroaryl, C₃-C₅ heterocycloalkyl optionally substituted with C(O), —(CH₂)ₐCO₂H optionally substituted with C₅-C₁₀ heteroaryl, (CH₂)ₐCONR³⁰R³¹, —(CH₂)ₐNHSO₃⁻, —(CH₂)ₐNHSO₃H, —(CH₂)ₐOH, —(CH₂)ₐOPO₃⁼, —(CH₂)ₐOPO₃H₂, —(CH₂)ₐOPO₃H⁻, —(CH₂)ₐOR²², —(CH₂)ₐOSO₃, —(CH₂)ₐOSO₃H, —(CH₂)ₐPO₃⁼, —(CH₂)ₐPO₃H₂, —(CH₂)ₐPO₃H⁻, —(CH₂)ₐSO₃⁻, —(CH₂)ₐSO₃H, —(CH₂)_{d}CO(CH₂CH₂O)_{c}R²³, —(CH₂)_{d}(CH₂CH₂O)_{c}R²⁴, —(CHCO₂H)ₐCO₂H, —CH₂(CHNH₂)ₐCH₂NR²⁵R²⁶, —CH₂(CHOH)ₐCO₂H, —CH₂(CHOH)ₐR²⁷, —CH[(CH₂)_{b}NH₂]ₐCH₂OH, —CH[(CH₂)_{b}NH₂]ₐCO₂H, and —(CH₂)ₐNR²⁸R²⁹;
each R²² to R³¹ are independently chosen from hydrogen, C₁-C₁₀ alkyl, and C₁-C₅-dicarboxylic acid;
R³⁵ is chosen from C₁-C₁₀ alkyl optionally substituted with hydroxyl and carboxylic acid, C₃-C₆ polyhydroxylated alkyl, C₅-C₁₀ aryl, C₅-C₁₀ heteroaryl, C₃-C₅ heterocycloalkyl optionally substituted with C(O), —(CH₂)ₐCO₂H optionally substituted with C₅-C₁₀ heteroaryl, —(CH₂)ₐCONR³⁰R³¹, —(CH₂)ₐNHSO₃⁻, —(CH₂)ₐNHSO₃H, —(CH₂)ₐOH, —(CH₂)ₐOPO₃⁼, —(CH₂)ₐOPO₃H₂, —(CH₂)ₐOPO₃H⁻, —(CH₂)ₐOR²², —(CH₂)ₐOSO₃⁻, —(CH₂)ₐOSO₃H, —(CH₂)ₐPO₃⁼, —(CH₂)ₐPO₃H₂, —(CH₂)ₐPO₃H⁻, —(CH₂)ₐSO₃⁻, —(CH₂)ₐSO₃H, —(CH₂)_{d}CO(CH₂CH₂O)_{c}R²³, —(CH₂)_{d}(CH₂CH₂O)_{c}R²⁴, —(CHCO₂H)ₐCO₂H, —CH₂(CHNH₂)ₐCH₂NR²⁵R²⁶, —CH₂(CHOH)ₐCO₂H, —CH₂(CHOH)ₐR²⁷, —CH[(CH₂)_{b}NH₂]ₐCH₂OH, —CH[(CH₂)_{b}NH₂]ₐCO₂H, and —(CH₂)ₐNR²⁸R²⁹; (AA) is a polypeptide chain comprising one or more natural or unnatural amino acids linked together by peptide bonds amide bonds and each instance of (AA) may be the same or different than each other instance, (PS) is a sulfated or non-sulfated polysaccharide chain comprising one or more monosaccharide units connected by glycosidic linkages; and each ‘a’, ‘b’, and ‘d’ are independently chosen from 0 to 10, ‘c’ is chosen from 1 to 100 and each of ‘m’ and ‘n’ independently is an integer from 1 to 3.

In some aspects, disclosed herein is a pyrazine compound of Formula I, or a pharmaceutically acceptable salt thereof, wherein: each of X¹ and X² is independently —CO₂R¹, —CONR¹R², —CO(AA) or —CONH(PS); each of Y¹ and Y² is independently selected from the group consisting of —NR¹R², Z¹ is a single bond, —CR¹R²—, —O—, —NR¹—, —NCOR¹—, —S—, —SO—, or —SO₂—; each of R¹ to R² are independently selected from the group consisting of H, —CH₂(CHOH)ₐH, —CH₂(CHOH)ₐCH₃, —CH₂(CHOH)ₐCO₂H, —(CHCO₂H)ₐCO₂H, —(CH₂CH₂O)_{c}H, —(CH₂CH₂O)_{c}CH₃, —(CH₂)ₐSO₃H, —(CH₂)ₐSO₃—, —(CH₂)ₐSO₂H, —(CH₂)ₐSO₂—, —(CH₂)ₐNHSO₃H, —(CH₂)ₐNHSO₃—, —(CH₂)ₐNHSO₂H, —(CH₂)ₐNHSO₂—, —(CH₂)ₐPO₄H₃, —(CH₂)ₐPO₄H₂⁻, —(CH₂)ₐPO₄H²⁻, —(CH₂)ₐPO₄³⁻, —(CH₂)ₐPO₃H₂, —(CH₂)ₐPO₃H⁻, and —(CH₂)ₐPO₃²⁻; AA is a peptide chain comprising one or more amino acids selected from the group consisting of natural and unnatural amino acids, linked together by peptide or amide bonds and each instance of AA may be the same or different than each other instance; PS is a sulfated or non-sulfated polysaccharide chain comprising one or more monosaccharide units connected by glycosidic linkages; and ‘a’ is a number from 0 to 10, ‘c’ is a number from 1 to 100, and each of ‘m’ and ‘n’ are independently a number from 1 to 3. In another aspect, ‘a’ is a number from 1 to 10. In still yet another aspect, ‘a’ is 1, 2, 3, 4, 5, 6, 7, 8, 9 or 10.

In some aspects, at least one of X¹ and X² is —CO(PS) or —CO(AA). In yet another aspect, both X¹ and X² are —CO(AA).

(AA) is a peptide chain comprising one or more natural or unnatural amino acids linked together by peptide or amide bonds. The peptide chain (AA) may be a single amino acid, a homopolypeptide chain or a heteropolypeptide chain, and may be any appropriate length. In some embodiments, the natural or unnatural amino acid is an α-amino acid. In yet another aspect, the α-amino acid is a D-α-amino acid or an L-α-amino acid. In a polypeptide chain comprising two or more amino acids, each amino acid is selected independently of the other(s) in all aspects, including, but not limited to, the structure of the side chain and the stereochemistry. For example, in some embodiments, the peptide chain may include 1 to 100 amino acid(s), 1 to 90 amino acid(s), 1 to 80 amino acid(s), 1 to 70 amino acid(s), 1 to 60 amino acid(s), 1 to 50 amino acid(s), 1 to 40 amino acid(s), 1 to 30 amino acid(s), 1 to 20 amino acid(s), or even 1 to 10 amino acid(s). In some embodiments, the peptide chain may include 1 to 100 α-amino acid(s), 1 to 90 α-amino acid(s), 1 to 80 α-amino acid(s), 1 to 70 α-amino acid(s), 1 to 60 α-amino acid(s), 1 to 50 α-amino acid(s), 1 to 40 α-amino acid(s), 1 to 30 α-amino acid(s), 1 to 20 α-amino acid(s), or even 1 to 10 α-amino acid(s). In some embodiments, the amino acid is selected from the group consisting of D-alanine, D-arginine D-asparagine, D-aspartic acid, D-cysteine, D-glutamic acid, D-glutamine, glycine, D-histidine, D-homoserine, D-isoleucine, D-leucine, D-lysine, D-methionine, D-phenylalanine, D-proline, D-serine, D-threonine, D-tryptophan, D-tyrosine, and D-valine. In some embodiments, the α-amino acids of the peptide chain (AA) are selected from the group consisting of arginine, asparagine, aspartic acid, glutamic acid, glutamine, histidine, homoserine, lysine, and serine. In some embodiments, the α-amino acids of the peptide chain (AA) are selected from the group consisting of aspartic acid, glutamic acid, homoserine and serine. In some embodiments, the peptide chain (AA) refers to a single amino (e.g., D-aspartic acid or D-serine).

In some embodiments, (AA) is a single amino acid selected from the group consisting of the 21 essential amino acids. In other aspects, AA is selected from the group consisting of D-arginine, D-asparagine, D-aspartic acid, D-glutamic acid, D-glutamine, D-histidine, D-homoserine, D-lysine, and D-serine. Preferably, AA is D-aspartic acid, glycine, D-serine, or D-tyrosine. Most preferably, AA is D-serine.

In some embodiments, (AA) is a β-amino acid. Examples of 3-amino acids include, but are not limited to, β-phenylalanine, β-alanine, 3-amino-3-(3-bromophenyl)propionic acid, 3-aminobutanoic acid, cis-2-amino-3-cyclopentene-1-carboxylic acid, trans-2-amino-3-cyclopentene-1-carboxylic acid, 3-aminoisobutyric acid, 3-amino-2-phenylpropionic acid, 3-amino-4-(4-biphenylyl)butyric acid, cis-3-amino-cyclohexanecarboxylic acid, trans-3-amino-cyclohexanecarboxylic acid, 3amino-cyclopentanecarboxylic acid, 3-amino-2-hydroxy-4-phenylbutyric acid, 2-(aminomethyl)phenylacetic acid, 3-amino-2-methylpropionic acid, 3-amino-4-(2-naphthyl)butyric acid, 3-amino-5-phenylpentanoic acid, 3-amino-2-phenylpropionic acid, 4-bromo-β-Phe-OH, 4-chloro-β-Homophe-OH, 4-chloro-β-Phe-OH, 2-cyano-β-Homophe-OH, 2-cyano-β-Homophe-OH, 4-cyano-β-Homophe-OH, 3-cyano-3-Phe-OH, 4-cyano-β-Phe-OH, 3,4-dimethoxy-β-Phe-OH, γ,γ-diphenyβ-Homoala-OH, 4-fluoro-3-Phe-OH, β-Gln-OH, β-Homoala-OH, β-Homoarg-OH, β-Homogln-OH, β-Homoglu-OH, β-Homohyp-OH, β-Homoleu-OH, β-Homolys-OH, β-Homomet-OH, β2-homophenylalanine, β-Homophe-OH, β3-Homopro-OH, β-Homoser-OH, β-Homothr-OH, β-Homotrp-OH, β-Homotrp-OMe, β-Homotyr-OH, β-Leu-OH, β-Leu-OH, β-Lys(Z)—OH, 3-methoxy-β-Phe-OH, 3-methoxy-β-Phe-OH, 4-methoxy-β-Phe-OH, 4-methy-β-Homophe-OH, 2-methyl-β-Phe-OH, 3-methyl-β-Phe-OH, 4-methyl-β-Phe-OH, β-Phe-OH, 4-(4-pyridyl)-β-Homoala-OH, 2-(trifluoromethyl)-β-Homophe-OH, 3-(trifluoromethyl)-β-Homophe-OH, 4-(trifluoromethyl)-β-Homophe-OH, 2-(trifluoromethyl)-3-Phe-OH, 3-(trifluoromethyl)-β-Phe-OH, 4-(trifluoromethyl)-β-Phe-OH, β-Tyr-OH, Ethyl 3-(benzylamino)propionate, β-Ala-OH, 3-(amino)-5-hexenoic acid, 3-(amino)-2-methylpropionic acid, 3-(amino)-2-methylpropionic acid, 3-(amino)-4-(2-naphthyl)butyric acid, 3,4-difluoro-β-Homophe-OH, γ,γ-diphenyl-β-Homoala-OH, 4-fluoro-β-Homophe-OH, β-Gln-OH, β-Homoala-OH, β-Homoarg-OH, β-Homogln-OH, β-Homoglu-OH, β-Homohyp-OH, β-Homoile-OH, β-Homoleu-OH, β-Homolys-OH, β-Homomet-OH, β-Homophe-OH, β3-homoproline, β-Homothr-OH, β-Homotrp-OH, β-Homotyr-OH, β-Leu-OH, 2-methyl-β-Homophe-OH, 3-methyl-β-Homophe-OH, β-Phe-OH, 4-(3-pyridyl)-β-Homoala-OH, 3-(trifluoromethyl)-β-Homophe-OH, β-Glutamic acid, β-Homoalanine, β-Homoglutamic acid, β-Homoglutamine, β-Homohydroxyproline, β-Homoisoleucine, β-Homoleucine, β-Homomethionine, β-Homophenylalanine, β-Homoproline, β-Homoserine, β-Homothreonine, β-Homotryptophan, β-Homotyrosine, β-Leucine, 13-Phenylalanine, Pyrrolidine-3-carboxylic acid and β-Dab-OH.

(PS) is a sulfated or non-sulfated polysaccharide chain including one or more monosaccharide units connected by glycosidic linkages. The polysaccharide chain (PS) may be any appropriate length. For instance, in some embodiments, the polysaccharide chain may include 1 to 100 monosaccharide unit(s), 1 to 90 monosaccharide unit(s), 1 to 80 monosaccharide unit(s), 1 to 70 monosaccharide unit(s), 1 to 60 monosaccharide unit(s), 1 to 50 monosaccharide unit(s), 1 to 40 monosaccharide unit(s), 1 to 30 monosaccharide unit(s), 1 to 20 monosaccharide unit(s), or even 1 to 10 monosaccharide unit(s). In some embodiments, the polysaccharide chain (PS) is a homopolysaccharide chain consisting of either pentose or hexose monosaccharide units. In other embodiments, the polysaccharide chain (PS) is a heteropolysaccharide chain consisting of one or both pentose and hexose monosaccharide units. In some embodiments, the monosaccharide units of the polysaccharide chain (PS) are selected from the group consisting of glucose, fructose, mannose, xylose and ribose. In some embodiments, the polysaccharide chain (PS) refers to a single monosaccharide unit (e.g., either glucose or fructose). In yet another aspect, the polysaccharide chain is an amino sugar where one or more of the hydroxy groups on the sugar has been replaced by an amine group. The connection to the carbonyl group can be either through the amine or a hydroxy group.

In some embodiments, for the pyrazine compound of Formula I, at least one of either Y¹ or Y² is

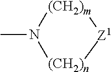

Z¹ is a single bond, —CR¹⁸R¹⁹—, —O—, —NR²⁰—, —NCOR²¹—, —S—, —SO—, and —SO₂—;

each R¹ to R²¹ are independently chosen from hydrogen, C₁-C₁₀ alkyl optionally substituted with hydroxyl and carboxylic acid, C₃-C₆ polyhydroxylated alkyl, C₅-C₁₀ aryl, C₅-C₁₀ heteroaryl, C₃-C₅ heterocycloalkyl optionally substituted with C(O), —(CH₂)ₐCO₂H optionally substituted with C₅-C₁₀ heteroaryl, (CH₂)ₐCONR³⁰R³¹, —(CH₂)ₐNHSO₃⁻, —(CH₂)ₐNHSO₃H, —(CH₂)ₐOH, —(CH₂)ₐOPO₃⁼, —(CH₂)ₐOPO₃H₂, —(CH₂)ₐOPO₃H⁻, —(CH₂)ₐOR²², —(CH₂)ₐOSO₃, —(CH₂)ₐOSO₃H, —(CH₂)ₐPO₃a, —(CH₂)ₐPO₃H₂, —(CH₂)ₐPO₃H⁻, —(CH₂)ₐSO₃, —(CH₂)ₐSO₃H, —(CH₂)_{d}CO(CH₂CH₂O)_{c}R²³, —(CH₂)_{d}(CH₂CH₂O)_{c}R²⁴, —(CHCO₂H)ₐCO₂H, —CH₂(CHNH₂)ₐCH₂NR²⁵R²⁶, —CH₂(CHOH)ₐCO₂H, —CH₂(CHOH)ₐR²⁷, —CH[(CH₂)_{b}NH₂]ₐCH₂OH, —CH[(CH₂)_{b}NH₂]ₐCO₂H, and —(CH₂)ₐNR²⁸R²⁹;

each R²² to R³¹ are independently chosen from hydrogen, C₁-C₁₀alkyl, and C₁-C₅-dicarboxylic acid; R³⁵ is chosen from C₁-C₁₀ alkyl optionally substituted with hydroxyl and carboxylic acid, C₃-C₆ polyhydroxylated alkyl, C₅-C₁a aryl, C₅-C₁₀ heteroaryl, C₃-C₅ heterocycloalkyl optionally substituted with C(O), —(CH₂)ₐCO₂H optionally substituted with C₅-C₁₀ heteroaryl, —(CH₂)ₐCONR³⁰R³¹, —(CH₂)ₐNHSO₃⁻, —(CH₂)ₐNHSO₃H, —(CH₂)ₐOH, —(CH₂)ₐOPO₃, —(CH₂)ₐOPO₃H₂, —(CH₂)ₐOPO₃H⁻, —(CH₂)ₐOR²², —(CH₂)ₐOSO₃⁻, —(CH₂)ₐOSO₃H, —(CH₂)ₐPO₃⁼, —(CH₂)ₐPO₃H₂, —(CH₂)ₐPO₃H⁻, —(CH₂)ₐSO₃⁻, —(CH₂)ₐSO₃H, —(CH₂)_{d}CO(CH₂CH₂O)_{c}R²³, —(CH₂)_{d}(CH₂CH₂O)_{c}R²⁴, —(CHCO₂H)ₐCO₂H, —CH₂(CHNH₂)ₐCH₂NR²⁵R²⁶, —CH₂(CHOH)ₐCO₂H, —CH₂(CHOH)ₐR²⁷, —CH[(CH₂)_{b}NH₂]ₐCH₂OH, —CH[(CH₂)_{b}NH₂]ₐCO₂H, and —(CH₂)ₐNR²⁸R²⁹; (AA) is a polypeptide chain comprising one or more natural or unnatural amino acids linked together by peptide bonds amide bonds and each instance of (AA) may be the same or different than each other instance; (PS) is a sulfated or non-sulfated polysaccharide chain comprising one or more monosaccharide units connected by glycosidic linkages; -; a, c, m and n are as describe elsewhere herein.

In yet another aspect, at least one of Y¹ and Y² is —NR¹²R¹³, and R¹² to R¹³ are as described above. In yet another aspect, both Y¹ and Y² are —NR¹²R¹³ and R¹² to R¹³ are as described above. Alternatively, R¹² and R¹³ are both independently selected from the group consisting of H, —CH₂(CHOH)ₐCH₃, —(CH₂)ₐSO₃H, —(CH₂)ₐNHSO₃H, and —(CH₂)ₐPO₃H₂. In yet another aspect, both R¹² and R¹³ are hydrogen.

In yet another aspect, the pyrazine compound of Formula I is a compound of Table A. In an exemplary embodiment, the pyrazine compound of Formula I is MB-102 or MB-404. Methods for synthesizing the compounds of Table A are detailed in US Publication Number 20190125901 A1, U.S. Pat. Nos. 8,115,000, and 10,525,149, the disclosures of which are hereby incorporated by reference.

TABLE A

| Tracer | Molecular | | |
|---|---|---|---|
| Agent | Weight | | |
| Name | (Da) | Structure | Chemical Name |
| MB- 102 | 372 | 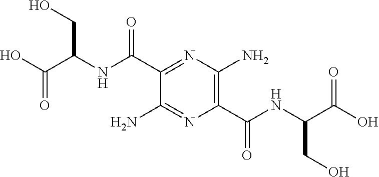 [SMILES: Nc1nc(C(=O)N[C@H](CO)C(=O)O)c(N)nc1C(=O)N[C@H](CO)C(=O)O] | 3,6-diamino- 2,5-bis{N-[(1R)- 1-carboxy-2- hydroxyethyl] carbamoyl}pyrazine |
| MB- 404 | 492 | 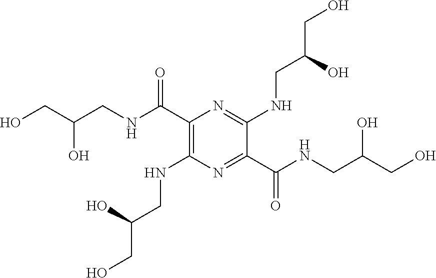 [SMILES: O=C(NCC(O)CO)c1nc(NC[C@H](O)CO)c(C(=O)NCC(O)CO)nc1NC[C@H](O)CO] | N²,N⁵-bis(2,3- dihydroxypropyl)- 3,6-bis[(S)-2,3- dihydroxypropylamino] pyrazine- 2,5-dicarboxamide |
| MB- 106 | 524 | 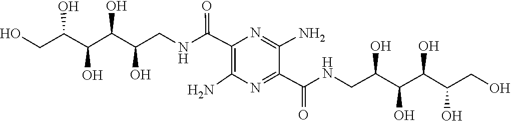 [SMILES: Nc1nc(C(=O)NC[C@@H](O)[C@H](O)[C@@H](O)[C@@H](O)CO)c(N)nc1C(=O)NC[C@@H](O)[C@H](O)[C@@H](O)[C@@H](O)CO] | 3,6-diamino-N²,N⁵- bis((2R,3S,4S,5S)- 2,3,4,5,6- pentahydroxyhexyl) pyrazine-2-5- dicarboxamide |
| MB- 216 | 2367 | 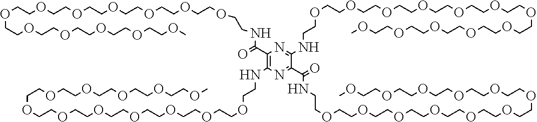 [SMILES: COCCOCCOCCOCCOCCOCCOCCOCCOCCOCCOCCOCCNC(=O)c1nc(NCCOCCOCCOCCOCCOCCOCCOCCOCCOCCOCCOCCOC)c(C(=O)NCCOCCOCCOCCOCCOCCOCCOCCOCCOCCOCCOCCOC)nc1NCCOCCOCCOCCOCCOCCOCCOCCOCCOCCOCCOCCOC] | 3,6-Bis(2,5,8,11, 14,17,20,23, 26,29,32,35- dodecaoxaheptatriacontan- 37-ylamino)-N²,N⁵- di(2,5,8,11,14, 17,20,23,26,29,32,35- dodecaoxaheptatriacontan- |
| | | | 37-yl)pyrazine-2,5- |
| | | | dicarboxamide |
| MB- 212 | 2395 | 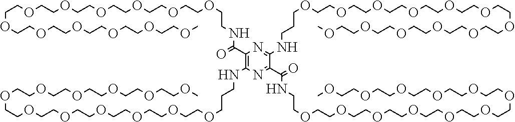 [SMILES: COCCOCCOCCOCCOCCOCCOCCOCCOCCOCCOCCOCCCNc1nc(C(=O)NCCOCCOCCOCCOCCOCCOCCOCCOCCOCCOCCOCCOC)c(NCCCOCCOCCOCCOCCOCCOCCOCCOCCOCCOCCOCCOC)nc1C(=O)NCCOCCOCCOCCOCCOCCOCCOCCOCCOCCOCCOCCOC] | 3,6-Bis(2,5,8,11, 14,17,20,23, 26,29,32,35- dodecaoxaoctatriacontan- 38-ylamino)-N²,N⁵- di(2,5,8,11,14, 17,20,23,26,29,32,35- dodecaoxaheptatriacontan- |
| | | | 37-yl)pyrazine-2,5- |
| | | | dicarboxamide |
| MB- 116 | 2250 | 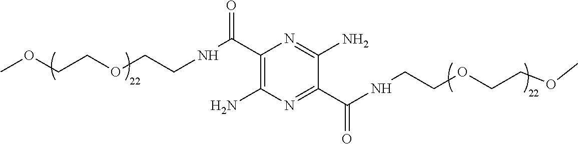 | 3,6-Bis(2,5,8,11,14, 17,20,23,26,29, 32,35,38,41,44,47, 50,53,56,59,62,65,68- tricosaoxaheptacontan- 70-yl)pyrazine-2,5- dicarboxamide |
| MB- 206 | 520 | 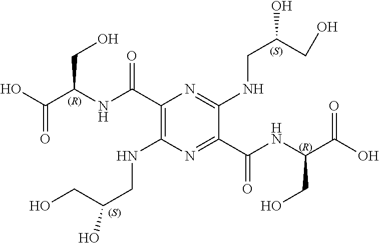 | D-Serine,N,N′- [[3,6-bis[[(2S)- 2,3-dihydroxypropyl] amino]-2,5-pyrazinediyl] dicarbonyl]bis- |
| MB- 112 | 2339 | 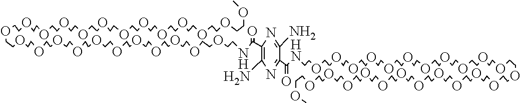 [SMILES: COCCOCCOCCOCCOCCOCCOCCOCCOCCOCCOCCOCCOCCOCCOCCOCCOCCOCCOCCOCCOCCOCCOCCOCCNC(=O)c1nc(N)c(C(=O)NCCOCCOCCOCCOCCOCCOCCOCCOCCOCCOCCOCCOCCOCCOCCOCCOCCOCCOCCOCCOCCOCCOCCOCCOC)nc1N] | 3,6-diamino- N²,N⁵-di(2,5,8, 11,14,17,20,23,26,29, 32,35,38,41,44,47,50, 53,56,59,62,65,68,71- tetracosaoxatriheptacontan- 73-yl)pyrazine-2,5- |
| | | | dicarboxamide |
| MB- 402 | 344 | 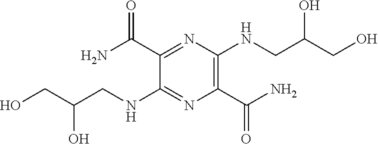 [SMILES: NC(=O)c1nc(NCC(O)CO)c(C(N)=O)nc1NCC(O)CO] | 3,6-N,N′-Bis(2,3- dihydroxypropyl)-2,5- pyrazinedicarboxamide |

Pharmaceutically acceptable salts are known in the art. In any aspect herein, the pyrazine may be in the form of a pharmaceutically acceptable salt. By way of example and not limitation, pharmaceutically acceptable salts include those as described by Berge, et al. in J. Pharm. Sci., 66(1), 1 (1977), which is incorporated by reference in its entirety for its teachings thereof. The salt may be cationic or anionic. In some embodiments, the counter ion for the pharmaceutically acceptable salt is selected from the group consisting of acetate, benzenesulfonate, benzoate, besylate, bicarbonate, bitartrate, bromide, calcium edetate, camsylate, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate, diphosphate, polygalacturonate, salicylate, stearate, subacetate, succinate, sulfate, tannate, tartrate, teoclate, triethiodide, adipate, alginate, aminosalicylate, anhydromethylenecitrate, arecoline, aspartate, bisulfate, butylbromide, camphorate, digluconate, dihydrobromide, disuccinate, glycerophosphate, jemisulfate, judrofluoride, judroiodide, methylenebis(salicylate), napadisylate, oxalate, pectinate, persulfate, phenylethylbarbarbiturate, picrate, propionate, thiocyanate, tosylate, undecanoate, benzathine, chloroprocaine, choline, diethanolamine, ethylenediamine, meglumine, procaine, benethamine, clemizole, diethylamine, piperazine, tromethamine, aluminum, sodium, calcium, lithium, magnesium, potassium, sodium zinc, barium and bismuth. Any functional group in the pyrazine compound capable of forming a salt may optionally form one using methods known in the art. By way of example and not limitation, amine hydrochloride salts may be formed by the addition of hydrochloric acid to the pyrazine. Phosphate salts may be formed by the addition of a phosphate buffer to the pyrazine. Any acid functionality present, such as a sulfonic acid, a carboxylic acid, or a phosphonic acid, may be deprotonated with a suitable base and a salt formed. Alternatively, an amine group may be protonated with an appropriate acid to form the amine salt. The salt form may be singly charged, doubly charged or even triply charged, and when more than one counter ion is present, each counter ion may be the same or different than each of the others.

### II. Compositions

Also disclosed herein are compositions for subcutaneous or intramuscular administration comprising a pyrazine compound of Formula I or pharmaceutically acceptable salt thereof, and a pharmaceutically acceptable excipient.

Suitable pyrazine compounds of Formula I and pharmaceutically acceptable salts thereof are described in detail in Section I. In some embodiments, the pyrazine compound of Formula I or pharmaceutically acceptable salt thereof is a compound of Table A. In a specific example, the compound is MB-102 or MB-404. The amount of a pyrazine compound of Formula I or pharmaceutically acceptable salt thereof in a composition of the present disclosure may be about 60 mg/mL to about 300 mg/mL. For instance, the amount of a pyrazine compound of Formula I or pharmaceutically acceptable salt thereof may be about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 330 mg/mL, about 440 mg/mL, about 250 mg/mL, about 260 mg/mL, about 270 mg/mL, about 280 mg/mL, about 290 mg/mL, or about 300 mg/mL. In some embodiments, the amount may be about 60 mg/mL to about 150 mg/mL, about 60 mg/mL to about 120 mg/mL, or about 60 mg/mL to about 100 mg/mL. In some embodiments, the amount may be about 60 mg/mL to about 90 mg/mL, or about 60 mg/mL to about 80 mg/mL.

Suitable pharmaceutically acceptable excipients are selected from the group consisting of solvents, pH adjusting agents, buffering agents, antioxidants, tonicity modifying agents, osmotic adjusting agents, preservatives, antibacterial agents, stabilizing agents, viscosity adjusting agents, surfactants and combinations thereof.

Pharmaceutically acceptable solvents may be aqueous or non-aqueous solutions, suspensions, emulsions, or appropriate combinations thereof. Non-limiting examples of non-aqueous solvents are propylene glycol, polyethylene glycol, vegetable oils such as olive oil, and injectable organic esters such as ethyl oleate. Examples of aqueous carriers are water, alcoholic/aqueous solutions, emulsions or suspensions, including saline and buffered media. By way of example and not limitation, pharmaceutically acceptable buffers include acetate, benzoate, carbonate, citrate, dihydrogen phosphate, gluconate, glutamate, glycinate, hydrogen phosphate, lactate, phosphate, tartrate, Tris-HCl, or combinations thereof having a pH of about 4 to about 9, preferably about pH 5 to about pH 8, most preferably about pH 6 to about pH 8, very most preferably about pH 7.0 to about pH 7.5. In yet another aspect, the pH is between 6.7 and 7.7. Other buffers, as are known in the art, may be selected based on the specific salt form of the pyrazine compound prepared or the specific medical application. A specific example, a buffer is phosphate buffered saline at physiological pH (approximately 7.2). Examples of the tonicity modifying agent are glycerol, sorbitol, sucrose, or, preferably, sodium chloride and/or mannitol. Examples of the viscosity adjusting agent include bentonite, calcium magnesium silicate and the like. Examples of the diluent include ethanol, methanol, water and the like. Examples of the antimicrobial include benzalkonium chloride, benzethonium chloride, ethylparaben, methylparaben and the like. Examples of osmotic adjusting agents include aminoethanol, calcium chloride, choline, dextrose, diethanolamine, lactated Ringer's solution, meglumine, potassium chloride, Ringer's solution, sodium bicarbonate, sodium chloride, sodium lactate, TRIS, or combinations thereof. These examples are for illustration only and are not intended to be exhaustive or limiting.

In some embodiments, a composition for subcutaneous or intramuscular administration may comprise a pyrazine compound of Formula I or pharmaceutically acceptable salt thereof and phosphate buffered saline. In some embodiments, a composition for subcutaneous or intramuscular administration may comprise a pyrazine compound of Formula I or pharmaceutically acceptable salt, sodium chloride, a dihydrogen phosphate salt (e.g., sodium dihydrogen phosphate monohydrate), water for injection. In each of the above embodiments, the pH is preferably about pH 6.7 to about pH 7.7, or about pH 7.0 to about pH 7.5, or about pH 7.2 to about pH 7.4.

Compositions of the present disclosure have a tonicity, pH and osmolarity suitable for administration to a patient by subcutaneous or intramuscular administration. The tonicity, pH and osmolarity of a composition may be adjusted using a tonicity adjusting agent, a buffer or other pH adjusting agent, or an osmolarity adjusting agent, respectively, by methods known in the art or detailed herein. Non-limiting examples of tonicity adjusting agents, buffers, other pH adjusting agents, and osmolarity adjusting agents are provided above.

Compositions of the present disclosure are typically stable against degradation and other adverse chemical reactions, and possesses a pharmaceutically-acceptable shelf-life. “Stable”, as used herein, means remaining in a state or condition that is suitable for administration to a patient (e.g., free of visible particulate matter, containing an amount of the pyrazine derivative within ±15% of the label claim, etc.). Formulations according to the present disclosure are found to be stable when maintained at about 4° C. to about 25° C. for at least 12 months, and are generally stable at about 4° C. for 12 to 24 months.

In some embodiments, compositions of the present disclosure may be a sterile composition. A “sterile” composition, as used herein, means a composition that has been brought to a state of sterility and has not been subsequently exposed to microbiological contamination, e.g., the container holding the sterile composition has not been compromised. Sterile compositions are generally prepared by pharmaceutical manufacturers in accordance with current Good Manufacturing Practice (“cGMP”) regulations of the U.S. Food and Drug Administration. In some embodiments, the composition is packaged in a sealed container and subjected to terminal sterilization to reduce or eliminate the microbiological burden of the formulation. The container may be any container suitable for use in a medical setting, examples include, but are not limited to, a vial, an ampule, a bag, a bottle and a syringe.

In some embodiments, the composition can take the form of a sterile, ready-to-use formulation for subcutaneous or intramuscular administration. This avoids the inconvenience of diluting a concentrated formulation into infusion diluents prior to injection, as well as reducing the risk of microbiological contamination during aseptic handling and any potential calculation or dilution error. Alternatively, the formulation may be a concentrated liquid formulation or solid formulation that is diluted prior to administration to the patient.

In an exemplary embodiment, the present disclosure provides an aqueous, sterile pharmaceutical composition for subcutaneous or intramuscular injection comprising about 60 mg/mL to about 300 mg/mL of a pyrazine compound of Formula I or a pharmaceutically acceptable salt thereof, about 0.01 to about 2 M buffering agent, about 0 mg/mL to about 500 mg/mL of an osmotic-adjusting agent, and from about 0 mg/mL to about 500 mg/mL of a tonicity-adjusting agent. Suitable buffering agents, osmotic-adjusting agents, and tonicity-adjusting agents are described above. The aqueous, sterile pharmaceutical composition may also optionally include one or more additional pharmaceutically acceptable excipients selected from those described above. The pH of the aqueous, sterile pharmaceutical composition is suitable for administration to a patient. In some embodiments, the pH is between 4 and 9, preferably between 5 and 8, most preferably between 6 and 8. In a specific example, the pH is about pH 6.7 to about pH 7.7. In another specific example, the pH is about pH 7.0 to about pH 7.5. In yet another specific example, the pH is about pH 7.2 to about pH 7.4. In some examples, the amount of the pyrazine compound of Formula I or a pharmaceutically acceptable salt thereof may be about 60 mg/mL to about 150 mg/mL, about 60 mg/mL to about 120 mg/mL, or about 60 mg/mL to about 100 mg/mL. In some example, the amount of the pyrazine compound of Formula I or a pharmaceutically acceptable salt thereof may be about 60 mg/mL to about 90 mg/mL, or about 60 mg/mL to about 80 mg/mL. In a specific example, the pyrazine compound of Formula I is MB-102 or MB-404.

The aqueous, sterile pharmaceutical composition disclosed herein is suitable for subcutaneous or intramuscular administration to a patient in need thereof. For example, the composition may be administered in the form of a bolus injection or several smaller injections. Ready-to-use formulations disclosed herein are preferably administered by bolus injection. In various embodiments, a ready-to-use formulation may be administered using auto-injector based administration. In other embodiments, the formulation may be self-administered by a patient as further described below. Typically, the volume of a ready-to-use formulation is about 0.5 mL to about 10 mL.

### III. Methods of Use

The present disclosure also provides a method for measuring organ function in a patient in need thereof. The method comprises subcutaneously or intramuscularly administering to said patient about 3 mg to about 250 mg of a pyrazine compound of Section I as a 60-300 mg/mL solution, or more preferably as a 60-150 mg/mL solution, wherein the administration produces a plasma concentration of the pyrazine compound, in the patient, that is substantially similar to the plasma concentration produced when an identical amount of the pyrazine compound is administered intravenously; measuring a concentration of the pyrazine compound in said patient, and determining organ function from the measurement. Determining organ function from the measurement may involve comparison of the patient's measurement of the pyrazine compound to a measurement obtained from a healthy control administered the same pyrazine compound under the same conditions. Alternatively, or in addition, determining organ function from the measurement may involve comparison of the patient's measurement of the pyrazine compound to an earlier measurement obtained from the patient under the same conditions (e.g., days, weeks, months or years earlier, optionally at a time before a treatment). In preferred embodiments, the solution comprising the pyrazine compound is a composition of Section II, more preferably an aqueous sterile pharmaceutical composition disclosed therein. In various embodiments, the organ may be a kidney, eye, or an intestine. In some embodiments, the organ is a kidney and the method provides a measurement of renal function. In some embodiments, the organ is an intestine and the method provides a measurement of gastrointestinal permeability. In some embodiments, the organ is an eye and the method provides a measurement of ocular angiography.

In a specific embodiment, the present disclosure provides a method for measuring renal glomerular filtration rate (GFR) in a patient in need thereof. The method comprises subcutaneously or intramuscularly administering to said patient about 3 mg to about 250 mg of a pyrazine compound of Section I as a 60-300 mg/mL solution, or more preferably as a 60-150 mg/mL solution, wherein the administration produces a plasma concentration of the pyrazine compound, in the patient, that is substantially similar to the plasma concentration produced when an identical amount of the pyrazine compound is administered intravenously; measuring a concentration of the pyrazine compound in said patient, and determining GFR from the measurement. In preferred embodiments, the solution comprising the pyrazine compound is a composition of Section II, more preferably an aqueous sterile pharmaceutical composition disclosed therein.

The total amount of the pyrazine compound administered can vary. In some embodiments, the total amount of the pyrazine compound administered may be about 10 mg to about 150 mg, about 50 mg to about 250 mg, or about 60 mg to about 160 mg. In some embodiments, the total amount of the pyrazine compound administered may be about 100 mg to about 300 mg, about 100 mg to about 200 mg, about 100 mg to about 150 mg, about 110 mg to about 160 mg, about 120 mg to about 170 mg, or about 130 mg to about 180 mg. In some embodiments, the total amount of the pyrazine compound administered may be about 3 mg to about 100 mg, about 3 mg to about 75 mg, about 3 mg to about 50 mg, or about 3 mg to about 30 mg. In some embodiments, the total amount of the pyrazine compound administered may be about 3 mg to about 20 mg, about 3 mg to about 15 mg, or about 3 mg to about 10 mg. A suitable amount of the pyrazine compound may be determined by methods known in the art. In some embodiments, a suitable amount of the pyrazine compound may be determined based on the patient's weight. For example, a suitable amount may be about 0.5 mg/kg to about 5.0 mg/kg, about 0.5 mg/kg to about 1.5 mg/kg, or about 1.0 mg/kg to about 1.5 mg/kg.

The pyrazine compound is administered is typically administered as a 60-300 mg/mL solution. Solution with higher concentrations of the pyrazine compound may be administered but will be more viscous. Higher viscosity may be addressed by using a larger bore needle than is typically needed for intramuscular or subcutaneous administration.

Whether administered subcutaneously or intramuscularly, the total amount of the pyrazine compound may be delivered via a single bolus injection or several smaller injections. In embodiments where the pyrazine compound is administered intramuscularly, the site of administration may include, but is not limited to, the deltoid or the gluteal muscle. In embodiments where the pyrazine compound is administered subcutaneously, the site of administration may include, but is not limited to the upper outer area of the arm, the front and outer sides of the thighs, the upper outer area of the buttocks, and the abdomen.

An injector device may be used to administer subcutaneously or intramuscularly the pyrazine compound. The injector device is configured such that a patient is able to self-administer the pyrazine compound outside of a hospital or clinical setting. For example, the patient is able to administer the pyrazine compound while at home. In some aspects, the injector device comes preloaded with the pyrazine compound already loaded into the device. In some aspects, the pyrazine compound is in a dose cartridge or other container, and the patient is provided with instructions as to how to load the dose cartridge or container into the injector device. In some aspects, the injector device is designed so that the patient can self-administer the pyrazine compound subcutaneously or intramuscularly.

Various auto-injectors are known in the art, including but not limited to Inject-Ease™, BD Physioject™, BD Intevia™, and BD Liberatas™ from Becton-Dickinson, VIBEX™, Bigshot™, and Quickshot™ from Antares Pharma and may be used with the pre-filled syringed disclosed in Section II. In one aspect, the patient places the pre-filled syringe in the auto-injector, places the tip against the patient's skin, and then presses a button on the auto-injector to automatically deliver the needle through the skin. In one aspect, the patient can control the rate at which the compound is injected, from seconds to a few minutes. In another aspect, the pre-filled syringe is provided to the patient pre-installed in the auto-injector.

In yet another aspect, the patient inspects the auto-injector for any visible damage and is instructed not to use if it appears damaged or broken, or if cap is missing or not secure. The patient then checks the expiration date and is instructed not to use if expired. Next the patient inspects the composition through a viewing window in the auto-injector to verify that it is bright yellow and free of particles. The patient is instructed to not use if the liquid is cloudy or if particles are present. The patient is then instructed to wash their hands and prepare the injection site by wiping the injection site with an alcohol swab and allowing the site to dry on its own. The patient administers the injection by removing the cap from the auto-injector, positioning the auto-injector on the site of administration, placing the auto-injector at a 90° angle to the injection site. In another aspect, the patient places the auto-injector at a 45° angle to the injection site. The patient pushes down while supporting the site of administration with the opposite hand. When the dose has been completely delivered, the patient removes the auto-injector from injection site. The full dose of the composition will be delivered in approximately 10 minutes or less—for instance, about 30 seconds to about 10 minutes. In some examples, the full dose can be delivered in about 2 minutes or less.

The concentration of the administered pyrazine compound in the patient (e.g., in the patient's blood, fluid, and/or tissue) may be measured by a variety of methods known in the art. As a non-limiting example, the concentration of the pyrazine compound in the patient may be measured by quantifying transdermal fluorescence in the patient. Measurements of transdermal fluorescence can be used to quantify the concentration of the pyrazine compound in a variety of physiological spaces—e.g., in blood, fluid, tissue, etc. In another non-limiting example, the pyrazine compound may be measured by taking aliquots of blood from the patient and measuring the concentration of the pyrazine by HPLC or other methods as are known it the art. For instance, the pyrazine compound may comprise a detectable label (e.g., a radioisotope, etc.) that can be quantified. In yet another non-limiting example, the concentration of the pyrazine compound may be measured by collecting the urine of the patient over a period of time and measuring the concentration of the pyrazine compound in the urine to determine the rate in which the kidneys eliminate the compound from the body of the patient.

In exemplary embodiments, the concentration of the administered pyrazine compound in the patient is measured by quantifying transdermal fluorescence. This may include contacting a medical device with the skin of the patient wherein said medical device is configured to cause a fluorescent reaction in the pyrazine compound, and detecting said reaction. The medical device may contact the skin of the patient in any suitable location. Specific locations known to be suitable are the sternum, lower sternum, pectoralis major, occipital triangle, forehead, chin, upper hip, and lower hip. Other locations on a patient may be used as determined by convenience, medical device design, and/or medical necessity. In some aspects, this method uses the medical devices and systems disclosed elsewhere herein.

The concentration of the administered pyrazine compound in the patient may be measured at a single point in time or over a measurement time window, as more fully described in U.S. Publication No. 20190125902, the disclosures of which are hereby incorporated by reference.

In yet another aspect, disclosed herein is a method for measuring renal glomerular filtration rate (GFR) in a patient in need thereof. The method comprises subcutaneously or intramuscularly administering to said patient about 3 mg to about 300 mg of a pyrazine compound of Section I as a 60-300 mg/mL solution, or more preferably as a 60-150 mg/mL solution, wherein the administration produces a plasma concentration of the pyrazine compound, in the patient, that is substantially similar to the plasma concentration produced when an identical amount of the pyrazine compound is administered intravenously; measuring transdermal fluorescence in said patient over a Measurement Time Window; and determining the GFR in said patient. In some aspects, the GFR of a patient is determined using the system disclosed elsewhere herein. In preferred embodiments, the solution comprising the pyrazine compound is a composition of Section II, more preferably an aqueous sterile pharmaceutical composition disclosed therein.

In one aspect of the above-described methods where the concentration of the pyrazine compound in the patient is measured by quantifying transdermal fluorescence, a display is used to prompt the user to attach the sensor at one or more particular body sites. In one such embodiment, a touch-screen interface is used, and the user is instructed to touch a rendition of the body site location at which the sensor was attached, in order to move to a next step in the measurement setup process. This has the benefit of discouraging placement of the sensor on body sites that are not appropriate or optimal for the GFR determination.

In another aspect, the next step is setting the light source output levels and the detector gain levels. In one such aspect, the detector gain levels and light source levels are both initially set to a low state and then the light source levels are sequentially increased until a targeted signal level is achieved. In one embodiment, the light source is the excitation source for the fluorescent GFR agent, and the source drive current is increased until either a targeted fluorescence signal is achieved, or a predefined maximum current is reached. In the case that the maximum source current is reached without attaining the desired fluorescence signal level, the detector gain is then sequentially increased until either the targeted fluorescence signal is achieved, or the maximum detector gain setting is reached.

In other aspects, a mobile computing device, system, and method for quantifying transdermal fluorescence is used, as more fully described in U.S. Publication No. 20200237282, the disclosures of which are hereby incorporated by reference.

In some aspects, measurement of the diffuse reflectance of the skin is made in addition to measurement of fluorescence of the skin and GFR agent as more fully described in U.S. Publication No. 20190125902, the disclosures of which are hereby incorporated by reference. In such aspects, the diffuse reflectance signal may be used to determine the optimum source output and detector gain levels. In yet further aspects, diffuse reflectance measurements are made within the wavelength bands for excitation and emission of the fluorescent GFR agent. In such aspects, setting of the LED source levels and detector gains may be performed by using the diffuse reflectance instead of the fluorescence signal levels to guide the settings. In one such aspect, the target levels or the diffuse reflectance signals are between 15% and 35% of the signal level at which detector or amplifier saturation effects are observed. This provides head-room for signal fluctuations that may be associated with patient movement or other physiological variation. The described procedures for optimizing the light source output and/or detection gains have the benefit that they provide a means of compensating for physiological variations across different patients, or across different body sites on the same patient. In one aspect, a primary factor that is compensated is the melanin content of the skin. Other physiological factors that may require compensation include blood content, water content, and scattering within the tissue volume that is optically interrogated by the sensor. In another aspect, if the desired signal targets are not attained, the user is prevented from proceeding with the measurement. In this manner, the reporting of inaccurate results is prevented.

Once the desired signal levels have been successfully achieved, in another aspect, a baseline signal is recorded. In one such aspect, the stability of the baseline is assessed, such as by fitting a slope to the signal over time, and the baseline is not accepted as valid unless the slope over time is below a pre-determined threshold. In some aspects, a display instructs the user not to proceed with administration of the tracer agent (i.e., the pyrazine compound) until a stable baseline has been achieved. In this manner, measurement is prevented if the sensor has not been properly positioned or attached. In addition, the user may be prevented from proceeding with a measurement if the tracer agent (i.e., the pyrazine compound) from a prior injection has not cleared out of the body yet to a desired degree.

Once a stable baseline is acquired, in another aspect of the above-mentioned method, the tracer agent (i.e., the pyrazine compound) is injected into the patient. The tracer agent administration is automatically detected as a rapid increase in the transdermal fluorescence of the patient as measured by the one or more sensors. A predetermined threshold for the rate of change, absolute signal change, or relative signal change may be employed for this purpose. The automatic agent detection may be reported to the user on a display device, such as a touch-screen monitor. In another aspect, once the tracer agent is detected, a further threshold is used to determine if sufficient tracer agent is present to initiate a GFR measurement. In one such aspect, measurements of fluorescence (Fₘₑₐₛ) and diffuse reflectance (DR) are combined in a manner which reduces the influence of physiological variation on the combined result (herein referred to as the Intrinsic Fluorescence or IF), so that, for example, the influence of skin color on the measurement is compensated for. The sufficiency of the tracer agent is then assessed by comparing the IF to a pre-determined threshold. In some aspects the IF is determined by using a formula of the form:

$$I F = \frac{{F}_{meas}}{{DR}_{ex}^{kex} {DR}_{em}^{kem} {DR}_{em , filtered}^{kem , filtered}} \qquad Equation ( 1 )$$

where the subscripts on the DR terms refer to measurements collected within the tracer agent excitation (ex) and emission (em) wavelength bands, with both filtered and un-filtered detectors, and the superscripts on the DR terms are calibration coefficients that may be determined through analysis of data collected previously on human patients, animals, in vitro studies, simulations, or any combination thereof. In this manner, if insufficient tracer agent has been administered for an accurate GFR assessment, the medical professional administering the measurement may be provided the opportunity to administer additional tracer agent, or to discontinue the measurement.

Once the tracer agent (i.e., the pyrazine compound) has been administered, in another aspect, the equilibration of the tracer agent into the extracellular space is monitored. In one aspect, the Measurement Time Window does not start until it has been determined that equilibration is sufficiently complete. A fit to an exponential function may be used to assess equilibration progress. For example, the change in fluorescence intensity over time may be fit to a single exponential function, and only once the fitted time constant is stable, is equilibration deemed to be complete. In one such aspect, a running estimate of when the first GFR determination will become available is provided to the user. In another aspect, the user is prevented from proceeding to the measurement phase until and unless sufficient equilibration has been achieved. In one such aspect, the equilibration time is compared to a predetermined threshold, and if the equilibration time exceeds the threshold, the user is prevented from proceeding with GFR determination. In this manner, if the sensor is located in a site that is in poor exchange with the circulatory system, the assessment of GFR is prevented.

In some aspects, the Reporting Time Interval, Measurement Time Window, and/or Single Injection Reporting Period are based on the specific medical assessment being performed and may vary accordingly. For example, for patients with chronic kidney failure, a single GFR determination may be sufficient. However, for patients with or at risk of acute kidney failure, a real-time assessment or GFR trend provides great potential benefit. In some aspects said Reporting Time Interval will be approximately 15 minutes. In other aspects said Reporting Time Interval will be approximately 30 minutes, approximately one hour, approximately two hours, approximately three hours, approximately five hours, approximately eight hours, approximately 10 hours, approximately 12 hours, approximately 18 hours, approximately 24 hours, approximately 36 hours, approximately 48 hours, approximately 72 hours, approximately 96 hours, or approximately 168 hours. In some aspects the Reporting Time Interval will be between 15 minutes and 168 hours. In some aspects the Single Injection Reporting Period will be based on the clearance half-life of the pyrazine compound. Said clearance half-life can be either previously determined in said patient, estimated based on the medical condition of said patient, or determined transdermally using the methods described herein. In some aspects said Single Injection Reporting Period is one clearance half-life, two clearance half-lives, three clearance half-lives, four clearance half-lives, five clearance half-lives, six clearance half-lives, eight clearance half-lives, or ten clearance half-lives. The maximum Single Injection Reporting Period is such that the pyrazine is no longer detectable in the blood stream of said patient. “Undetectable” as used herein means that the concentration of the pyrazine is no longer detectable by the method used to make the determination. In some instances, when the detection level of the instrument makes this an extremely long time period (e.g., over one week), “undetectable” means that the concentration level has dropped below 0.39% (i.e., eight clearance half-lives). In yet another aspect, the Reporting Time Interval is between approximately 1 and 168 hours and all one hour increments in between.

Likewise, the Measurement Time Window may vary according to the specific medical needs of the patient and may vary accordingly. In some aspects it will be approximately 15 minutes. In other aspects said Measurement Time Window will be approximately 30 minutes, approximately one hour, approximately two hours, approximately three hours, approximately five hours, approximately eight hours, approximately 10 hours, approximately 12 hours, approximately 18 hours, approximately 24 hours, approximately 36 hours, approximately 48 hours, approximately 72 hours, approximately 96 hours, or approximately 168 hours. In some aspects the Measurement Time Window will be between 15 minutes and 168 hours. There may be one or a plurality of Measurement Time Windows during each Single Injection Reporting Period. In some aspects, the Single Injection Reporting Period is divided into multiple Measurement Time Windows where each Measurement Time Window is the same. In yet another aspect, the Single Injection Reporting Period is divided into multiple Measurement Time Windows where each Measurement Time Windows is selected independently of the others and may be the same or different than the other Measurement Time Windows.

The methods and system disclosed herein have the benefit of automatically adjusting for skin melanin content, such that the GFR determination is accurate across a wide range of skin types and levels of pigmentation. The Fitzpatrick scale is a numerical classification scheme for human skin color. It is widely recognized as a useful tool for dermatological research into human skin pigmentation. Scores range from type I (very fair skin with minimal pigmentation) to type VI (deeply pigmented and dark brown). The system and methods disclosed herein are suitable for use with all six categories of skin pigmentation on the Fitzpatrick scale. Specifically, the systems and methods disclosed herein are suitable for use with skin pigmentation of type I, type II, type III, type IV, type V and type VI.

Also disclosed herein is a method of assessing organ function in a patient in need thereof. The method comprises subcutaneously or intramuscularly administering to said patient about 3 mg to about 300 mg of a pyrazine compound of Section I as a 60-300 mg/mL solution, or more preferably as a 60-150 mg/mL solution, wherein the administration produces a plasma concentration of the pyrazine compound, in the patient, that is substantially similar to the plasma concentration produced when an identical amount of the pyrazine compound is administered intravenously; exposing said patient to electromagnetic radiation thereby causing spectral energy to emanate from the pyrazine compound; detecting the spectral energy emanated from the pyrazine compound; and assessing the organ function of the patient based on the detected spectral energy. In various embodiments, the organ may be a kidney, eye, or an intestine. In some embodiments, the organ is a kidney and the method provides a measurement of renal function. In some embodiments, the organ is an intestine and the method provides a measurement of gastrointestinal permeability. In some embodiments, the organ is an eye and the method provides a measurement of ocular angiography.

In some aspects, the pyrazine compound is not metabolized by the patient; instead it is entirely eliminated by renal excretion without being metabolized (e.g., no oxidation, glucuronidation or other conjugation). In some aspects, at least 95% of the pyrazine compound is not metabolized by the patient prior to renal excretion. In some aspects, at least 96% of the pyrazine compound I is not metabolized by the patient prior to renal excretion. In some aspects, at least 97% of the pyrazine compound is not metabolized by the patient prior to renal excretion. In some aspects, at least 98% of the pyrazine compound is not metabolized by the patient prior to renal excretion. In some aspects, at least 99% of the pyrazine compound is not metabolized by the patient prior to renal excretion. In some embodiments, said pyrazine compound is entirely eliminated by said patient in less than a predetermined period of time. In some aspects, assessing the renal function in a patient may also include determining the GFR in the patient.

In the above described methods, the pyrazine compound may be administered to a patient suspected or known to have at least one medical issue with their kidneys, and the methods disclosed herein may be used to determine the level of renal impairment or deficiency present in said patient. In some aspects, said patient has an estimated GFR (eGFR) or previously determined GFR of less than 110, less than 90, less than 60, less than 30, or less than 15. The eGFR of a patient is determined using standard medical techniques, and such methods are known in the art. Alternatively, the pyrazine compound may be administered to a patient that does not have or is not suspected to have medical issues with their kidneys. The GFR monitoring may be done as part of a general or routine health assessment of a patient or as a precautionary assessment.

As is known in the art, the rate in which a patient eliminates waste from their blood stream (i.e., clearance half-life) is dependent on the health and proper functioning of their renal system. “Entirely eliminated” as used in this context means that the level of they pyrazine in the blood stream has dropped below 0.39% (i.e., eight half-lives). The clearance half-life will depend on the GFR of the patient and slows greatly as the functioning of the renal system degrades due to illness, age or other physiological factors. In a patient with no known risk factors associated with CKD, having a normal GFR and/or a normal eGFR, the Single Injection Reporting Period is 24 hours. In some aspects, the Single Injection Reporting Period for a patient with a GFR or eGFR below 110 is 24 hours. For a patient with a GFR or eGFR below 90, the Single Injection Reporting Period is 24 hours. For a patient with a GFR or eGFR below 60, the Single Injection Reporting Period is 48 hours. For a patient with a GFR or eGFR below 30, the Single Injection Reporting Period is 48 hours. In some aspects, the Single Injection Reporting Period for a patient with a GFR or eGFR below 110 is equal to eight clearance half-lives. For a patient with a GFR or eGFR below 90, the Single Injection Reporting Period is equal to eight clearance half-lives. For a patient with a GFR or eGFR below 60, the Single Injection Reporting Period is equal to eight clearance half-lives. For a patient with a GFR or eGFR below 30, the Single Injection Reporting Period is equal to eight clearance half-lives.

Because an increase of protein concentration in the urine of a patient may suggest some manner of kidney impairment or deficiency, the methods disclosed herein are suitable for patients whose urinalysis shows an increase in protein levels. In some aspects, the patient has an increased level of protein in their urine as determined by standard medical tests (e.g., a dipstick test). By way of example and not limitation, the urinalysis of a patient may show an increase in albumin, an increase in creatinine, an increase in blood urea nitrogen (i.e., the BUN test), or any combination thereof.

Still referring to the above-mentioned method, the pyrazine compound is exposed to electromagnetic radiation such as, but not limited to, visible, ultraviolet and/or infrared light. This exposure of the pyrazine to electromagnetic radiation may occur at any appropriate time but preferably occurs while the pyrazine compound is located inside the body of the patient. Due to this exposure of the pyrazine to electromagnetic radiation, the pyrazine emanates spectral energy (e.g., visible, ultraviolet and/or infrared light) that may be detected by appropriate detection equipment. The spectral energy emanated from the pyrazine compound tends to exhibit a wavelength range greater than a wavelength range absorbed. By way of example but not limitation, if an embodiment of the pyrazine compound absorbs light of about 440 nm, the pyrazine compound may emit light of about 560 nm.

Detection of the pyrazine (or more specifically, the spectral energy emanating therefrom) may be achieved through optical fluorescence, absorbance or light scattering techniques. In some aspects, the spectral energy is fluorescence. In some embodiments, detection of the emanated spectral energy may be characterized as a collection of the emanated spectral energy and the generation of an electrical signal indicative of the collected spectral energy. The mechanism(s) utilized to detect the spectral energy from the pyrazine compound present in the body of a patient may be designed to detect only selected wavelengths (or wavelength ranges) and/or may include one or more appropriate spectral filters. Various catheters, endoscopes, ear clips, hand bands, head bands, surface coils, finger probes and other medical devices disclosed in U.S. Pat. Nos. 9,632,094, 10,548,521, 10,194,854, U.S. Publication No. 20200223805, and U.S. Application No. 63/029,927, the disclosures of which are hereby incorporated by reference, may be utilized to expose the pyrazine compound to electromagnetic radiation and/or to detect the spectral energy emanating therefrom. The device that exposes the pyrazine to electromagnetic radiation and detects the spectral energy emanated therefrom may be the same or different. That is, one or two devices may be used. The detection of spectral energy may be accomplished at one or more times intermittently or may be substantially continuous.

Renal function, or GFR, of the patient is determined based on the detected spectral energy. This is achieved by using data indicative of the detected spectral energy and generating an intensity/time profile indicative of a clearance of the pyrazine compound from the body of the patient. This profile may be correlated to a physiological or pathological condition. For example, the patient's clearance profiles and/or clearance rates may be compared to known clearance profiles and/or rates to assess the patient's renal function and to diagnose the patient's physiological condition. In the case of analyzing the presence of the pyrazine compound in bodily fluids, concentration/time curves may be generated and analyzed (preferably in real time) in order to assess renal function. Alternatively, the patient's clearance profile can be compared to one or more previously measured clearance profiles from the same patient to determine if the kidney function of said patient has changed over time. In some aspects, renal function assessment is done using the system disclosed elsewhere herein.

Physiological function can be assessed by: (1) comparing differences in manners in which normal and impaired cells or organs eliminate the pyrazine compound from the bloodstream; (2) measuring a rate of elimination or accumulation of the pyrazine in the organs or tissues of a patient; and/or (3) obtaining tomographic images of organs or tissues having the pyrazine associated therewith. For example, blood pool clearance may be measured non-invasively from surface capillaries such as those in an ear lobe or a finger, or it can be measured invasively using an appropriate instrument such as an endovascular catheter. Transdermal fluorescence can also be monitored non-invasively on the body of said patient. Many locations on the epidermis of a patient may be suitable for non-invasively monitoring the transdermal fluorescence. The site on the patient is preferably one where vasculature to tissue equilibrium occurs relatively quickly. Examples of suitable sites on a patient include, but are not limited to, the sternum, the lower sternum, pectoralis major, the occipital triangle, the forehead, the chin, the upper hip, and the lower hip. Accumulation of a pyrazine compound within cells of interest can be assessed in a similar fashion.

In some aspects, the pyrazine compound is administered to a patient wherein said patient has been previously diagnosed with at least Stage 1 CKD. In other aspects, said patient has been previously diagnosed with Stage 2 CKD, Stage 3 CKD, Stage 4 CKD or Stage 5 CKD. In yet another aspect, the patient has not yet been diagnosed with CKD but has one or more risk factors associated with CKD. In yet another aspect, the patient has no known risk factors for CKD.

Administration of the pyrazine compound is done by any suitable method based on the medical test being performed and the medical needs of the patient. Suitable methods are disclosed elsewhere herein.

### IV. Systems

Also disclosed herein is a system for determining the GFR or assessing the renal function in a patient in need thereof. The system comprises a computing device, a display device communicatively coupled to said computing device, a power supply that is operatively coupled to said computing device and maintains electrical isolation of the system from external power sources, one or more sensor heads (disclosed in Section III above) operatively coupled to said computing device, and at least one tracer agent configured to emit light when exposed to electromagnetic radiation. The computing device is configured to operate and control the sensor heads, record one or more light measurements sent from said sensor heads, and calculate the GFR of said patient based on said light measurements.

In some aspects, the one or more sensor heads comprise at least one source of electromagnetic radiation, generate and deliver electromagnetic radiation to the skin of said patient, detect and measure electromagnetic radiation emitted by said tracer agent, and transmit said measurement of electromagnetic radiation emitted by said tracer agent to said computing device. In a system with more than one sensor head, each sensor head may be the same or different and the electromagnetic radiation emitted therefrom may be the same or different. In some aspects the sensor heads are configured to attach to the skin of said patient. By way of example and not limitation, in a system with two sensor heads, one sensor head may emit and monitor one wavelength of electromagnetic radiation while the second sensor head may emit and monitor a different wavelength. This would enable the data to be compared to increase the accuracy of the GFR determination and the information available to the medical professional administering the assessment. In yet another nonlimiting example, in a system with two sensor heads, the two sensor heads are used to separate the local equilibration kinetics from the terminal phase kinetics. This enables a medical professional to determine when equilibration is complete and reduces artifacts due to local movement of the sensors.

In other aspects, a mobile computing device, system, and method for quantifying transdermal fluorescence is used, as disclosed in Section III.

The tracer agent is a pyrazine compound of Section I, and it is configured to be administered to said patient via subcutaneous or intramuscular administration. In some aspects, the tracer is configured to be eliminated by only glomerular filtration in the kidneys of said patient, and emit light that is detectable by said sensor heads when exposed to electromagnetic radiation. Preferably the tracer agent is a compound of Table A. Most preferably, the tracer agent is (2R,2′R)-2,2′-((3,6-diaminopyrazine-2,5-dicarbonyl)bis(azanediyl))bis (3-hydroxypropanoic acid) (also called MB-102 or 3,6-diamino-N²,N⁵-bis(D-serine)-pyrazine-2,5-dicarboxamide). In some aspects, the tracer agent is in a formulation suitable for administration to a patient in need thereof, as described in Section II.

The system for determining the GFR or assessing the renal function in a patient may be configured to carry out the methods disclosed herein on a patient in need thereof. The computing device in the system may be any standard computer having all of the capabilities implied therewith, specifically including, but not limited to, a permanent memory, a processor capable of complex mathematical calculations, a keyboard and/or a mouse for interacting with the computer, and a display communicatively coupled to the computing device. As such, the permanent memory of the computing device may store any information, programs and data necessary to carry out the functions of the system for determining the GFR or assessing the renal function in a patient. Such information, programs, and data may be standards and/or controls which may be used to compare transdermal fluorescence values collected by the one or more sensor heads to known values. In some aspects, the computing device may save results from a previous assessment or GFR determination in a patient so that results obtained at a later date may be compared. This would permit a medical professional to monitor the health of the kidneys of a patient over time. In some aspects, the computing device is a laptop computer. In some aspects, the display device includes a touch screen.

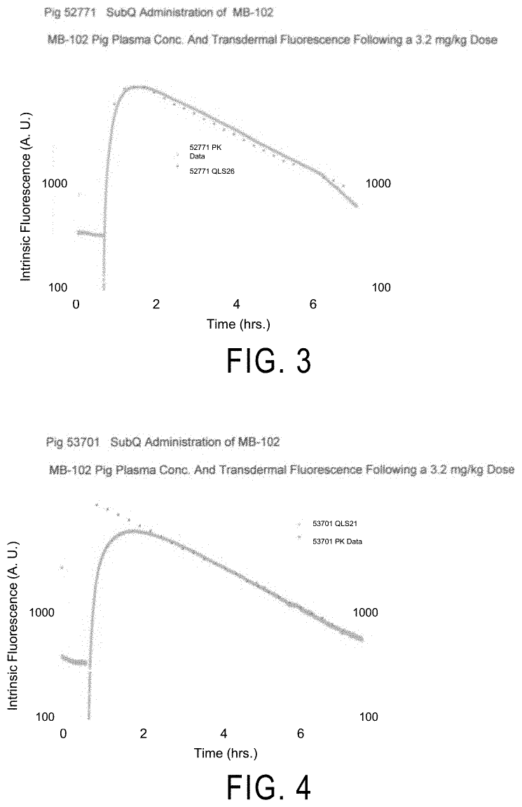

Additionally, the computing device is configured to calculate the time constant for renal decay over a predetermined period of time. In one aspect, the transdermal fluorescence data in a patient is collected over a predetermined period of time, and a graph is prepared of time (x-axis) versus fluorescence (y-axis). The rate of decay may be curved or linear and a time constant for the rate of decay is calculated. In one aspect, the rate of decay is linear for a semilog(y) plot. The time constant is compared to known values thereby determining the GFR in the patient. In some aspects, the rate of decay corresponds to standard first order kinetics. In yet another aspect, the rate of decay may exhibit a multi-compartment pharmacokinetic model. FIGS. 3A to 3D of US Publication Number 20190125901 A1 illustrate two-compartment pharmacokinetics by which standard pharmacokinetic software is able to determine the time constant for renal decay. GFR determination is done using linear regression, outlier exclusion, calculation of the correlation coefficient (R²) and standard error of calibration and more fully described in US Publication Number 20190125901 A1, the disclosures of which are hereby incorporated by reference.

### V. Kits

Also disclosed herein is a kit for GFR assessment. The kit may comprise about 3 mg to about 250 mg of a pyrazine compound of Section I as an about 60 mg/ml to an about 300 mg/ml solution as described in Section II; an injector device configured to subcutaneously or intramuscularly administer the solution into the body of a patient; sensor configured to attach to the body of the patient and detect transdermal fluorescence; a mobile computing device wirelessly communicatively coupled to the sensor and programmed to receive data from the sensor and calculate the GFR of the patient based on the data; and written instructions describing how to use the components of the kit in order to assess the GFR of the patient. Suitable injector devices and mobile computing devices are described in Section III.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1—MB-102 Formulation: Sodium Salt

MB-102 (0.300 g, 0.81 mmol), was placed in a vial (4.0 mL). Water was added (0.8 mL, D.I.) and the mixture sonicated for a brief period of time. To the mixture was added aqueous 6.25 N NaOH (2 equivalents, 1.62 mmol, 0.24 mL, added in portions; 0.020 mL×12 to effect solution) with stirring and brief periods of sonication. A deep red solution was obtained with no precipitate formation at 4° C. overnight. The final concentration was 300 mg (2R,2′R)-2,2′-((3,6-diaminopyrazine-2,5-dicarbonyl)bis(azanediyl))bis(3-hydroxypropanoic acid), MB-102 per mL water (w/v).

### Example 2—MB-102 Formulation: Sodium Salt

MB-102 (0.640 g, 1.72 mmol), was placed in a vial (20 mL scintillation) and 6.0 mL PBS-1× was added. The pH was adjusted to 7.2 by addition of 6.25 N NaOH. At this point all the di-acid MB-102 had dissolved. The total volume was adjusted to 10.0 mL by addition of PBS-1× for a final concentration of 64 mg per 1.0 mL solution.

### Example 3—MB-102 Formulation: Sodium Salt

The procedure of Example 2 was followed with the exception that the final volume was 5.0 mL resulting in a final concentration of 128 mg per 1.0 mL

### Solution

### Example 4—MB-102 Formulation: Choline Salt

MB-102 (0.30 g, 0.81 mmol) was weighed into a 5 mL v-vial equipped with a Teflon-covered magnetic spin vane. Choline hydroxide in water (Aldrich, 0.43 g of 46 wt %, 1.62 mmol) was added portion-wise with stirring until a solution was obtained. The total weight of the solution was 0.73 g or 64.2 wt % MB-102 di-choline salt (MW: 578.6). The solution was stored at 4° C. for 7 days with no precipitation.

### Example 5—MB-102 Formulation: Meglumine Salt

The di-meglumine salt of MB-102 was prepared using a procedure similar to that employed for Example 4 but substituting the appropriate quantities of meglumine for choline hydroxide.

### Example 6—General Animal Preparation and MB-102 Quantitation

Juvenile Yorkshire pigs weighing approximately 25 kg were anesthetized, their body temperature stabilized, and an IV port surgically placed. A region approximately half-way along the line between the hip and knee joint was selected as the injection site and prepared by cleaning the skin and removing any exposed hair by shaving the selected area at least twice. Similarly, the site where two Quantum Leap fluorescent sensors (disclosed in U.S. Pat. No. 10,548,521) were to be positioned (chest) was prepared by washing and shaving. After the skin was dry the sensors were attached with commercially available skin adhesive and background skin auto-fluorescence measurements were recorded. After about 20 minutes of background fluorescence collection the subject animal was dosed either intravenously (animal 48019) or subcutaneously (animals 52771, 53701 09802, or 1539) or intramuscularly (09801) with PBS-1× formulated MB-102 at 3.2 mg/kg. Injection of the appropriate volume took place over about a minute. Fluorescent skin signal at the chest was typically observed within several minutes post injection and increased over time until reaching equilibrium. At this point glomerular elimination of MB-102 by the kidneys led to a decrease of fluorescence signal over time. Blood samples (1.5 mL) were taken immediately prior to the introduction of MB-102 (0 minutes) and at 15 minute intervals throughout the procedure. Blood was collected in BD vacutainer K2EDTA tubes, spun to plasma and stored at −80° C. until analyzed. Subsequently, the processed plasma samples were thawed, diluted with PBS-1× and analyzed by HPLC or UPLC and the concentration of MB-102 determined using standard analytical techniques. The results for plasma concentration of MB-102 (ng/mL) versus time for several experiments are tabulated in Table 1. Further details for each animal are provided in the examples that follow.

MB-102 Dose Amount

48019 IV: 8.2 mL at 11.8 mg/mL

52771 SubQ: 1.2 mL at 60 mg/mL

53701 SubQ: 1.2 mL at 60 mg/mL

09802 SubQ: 1.3 mL at 60 mg/mL

09801 IM 1.4 mL at 60 mg/mL

TABLE 1
MB-102 Pig Plasma Conc. (ng/mL) Following a 3.2
mpk Dose (IV, Subcutaneous & IM)

| | 48019 | 52771 | 53701 | 09802 | 09801 |
|---|---|---|---|---|---|
| Time (Min.) | IV | SubQ | SubQ | SubQ | IM |
| 15 | 13048 | 6128 | 8106 | 6666 | 10025 |
| 30 | 9196 | 8511 | 7411 | 7528 | 8133 |
| 45 | 7830 | 8790 | 6621 | 6847 | 6243 |
| 60 | 5881 | 8845 | 5937 | 6543 | 5029 |
| 75 | 5433 | 7844 | 5257 | 5488 | 3925 |
| 90 | 4678 | 6922 | 4715 | 5262 | 3371 |
| 105 | 3998 | 6066 | 4231 | 4810 | 2725 |
| 120 | 3345 | 5560 | 3674 | 4414 | 2127 |
| 135 | 3251 | 4995 | 3254 | 3898 | 1805 |
| 150 | 2703 | 4405 | 3001 | 3707 | 1511 |
| 165 | 2410 | 3953 | 2639 | 3501 | 1344 |
| 180 | 2197 | 3476 | 2391 | 3068 | 1114 |
| 195 | 2002 | 3187 | 2168 | 2722 | 946 |
| 210 | 1912 | 2828 | 1939 | 2495 | 841 |
| 225 | 1689 | 2496 | 1671 | 2195 | 686 |
| 240 | 1514 | 2245 | 1511 | 1992 | 578 |
| 255 | 1404 | 1998 | 1351 | 1727 | 520 |
| 270 | 1322 | 1758 | 1225 | 1599 | 434 |
| 285 | 1138 | 1630 | 1088 | 1431 | 373 |
| 300 | 1106 | | 987 | 1295 | 333 |
| 315 | 1002 | | 867 | 1180 | 298 |
| 330 | 917 | 1206 | 800 | 1034 | 261 |
| 345 | 859 | 1139 | 704 | 914 | 222 |
| 360 | 819 | 1004 | 631 | 829 | 203 |
| SUM | 1,188,637 | 1,413,823 | 1,077,956 | 1,210,924 | 794,162 |
| (ng/mL*min) | | | | | |
| Dose (ng) | 96,800,000 | 72,000,000 | 72,000,000 | 78,000,000 | 84,000,000 |

### Example 7—IV Dosing of MB-102 (Pig)

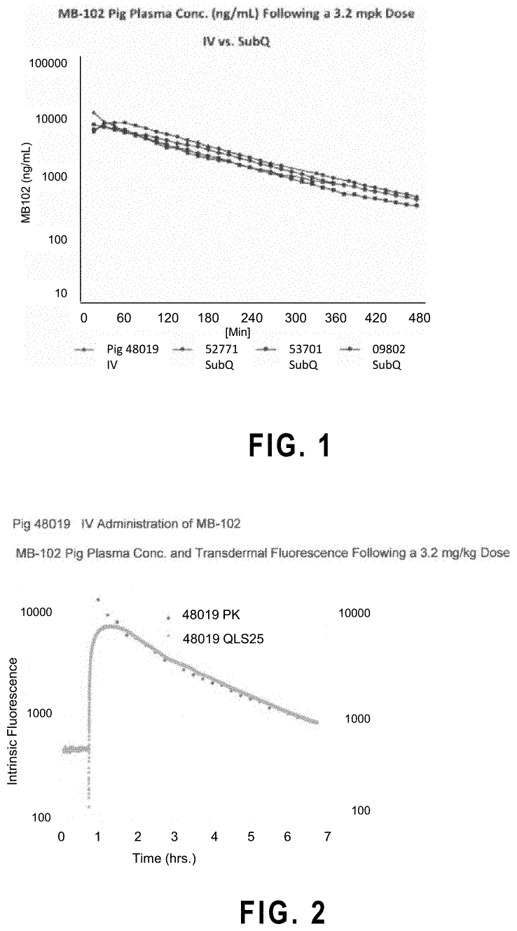

Subject pig, 48019, the control animal, was prepared as in Example 6 and dosed IV using 8.2 mL of 11.8 mg/mL MB-102 in PBS-1× (bolus over about 2 min). The results for MB-102 plasma concentration (ng/mL) versus time post IV injection is shown in Table 1 and plotted in FIG. 1. The corresponding transdermal fluorescence of MB-102 versus time post IV injection plot is shown in FIG. 2.

### Example 8—Multi-Needle Subcutaneous Dosing of MB-102 (Pig)

A sterile Mesoderm needle pack was obtained. The needle array featured five (5), 4 mm×30 gauge (0.30 mm dia) luer-hub needles arranged four (4) in a circular, symmetrical pattern (30 mm diameter) about a central needle and were attached via a distribution channel and a second luer-hub to a primary source syringe (3 mL in total deliverable volume). MB-102 API was formulated as 60 mg/mL of sodium salt in 1×PBS and placed in 5 mL serum cap vials. Immediately prior to dosing, the primary syringe was filled with a slight excess of the required amount of formulated MB-102 to achieve 3.2 mg/kg and the needle cap replaced until the subject animal and fluorescence monitor were ready for the experiment to begin and the injection initiated. Subject pig 52771 was dosed with 1.2 mL of MB-102 formulated in PBS-1× (3.2 mg/kg). Within a few minutes, transdermal fluorescent signal from MB-102 was detectable by the sensors referred to in Example 6. Subject animal fluorescence was obtained during the entire course of the experiment (8 hrs) and blood samples were collected every 15 minutes, processed and stored at −80° C. Comparison of IV MB-102 plasma concentration versus time and subcutaneous MB-102 (both at 3.2 mg/kg dose) plasma concentration versus time are shown in FIG. 1. The companion subcutaneous MB-102 transdermal fluorescence versus time plots are shown in FIG. 3.

### Example 9—Multi-Needle Subcutaneous Dosing of MB-102 (Pig)

Experimental animal Yorkshire pig, 53701, was subject to subcutaneous (subQ) administration of MB-102 as described in Example 8. The fluorescence signal was first observed after a few minutes. Plasma sampling was conducted as described in Example 8 and the results (Table 1) are plotted in FIG. 1. Corresponding companion transdermal fluorescence versus time results are shown in FIG. 4.

### Example 10—Single Needle Subcutaneous (subQ) Dosing of MB-102 (Pig)

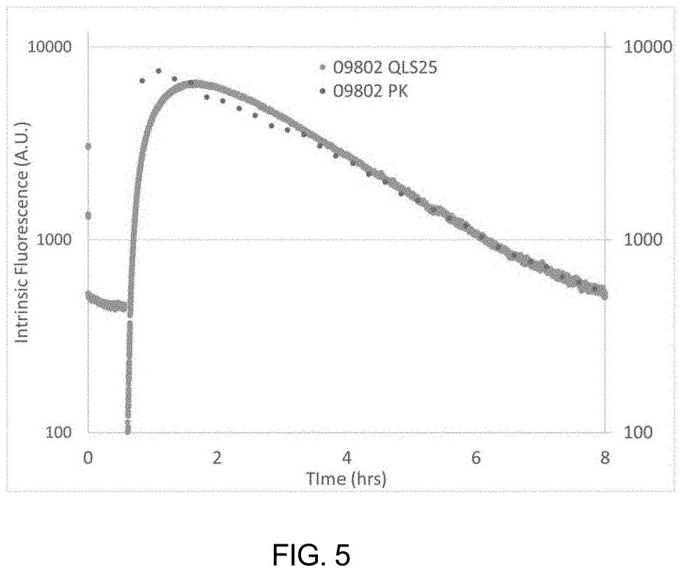

Experimental animal Yorkshire pig, 09802, was subject to subcutaneous (subQ) administration of MB-102 using a single needle in the same protocol as Example 8 but substituting a 2 mL syringe equipped with a single 4 mm×30 g needle. The fluorescence signal was first observed after a few minutes. Plasma sampling was conducted as described in Example 8 and the results (Table 1) are plotted in FIG. 1. Corresponding companion transdermal fluorescence versus time results are shown in FIG. 5.

### Example 11—Single Needle Intramuscular (IM) Dosing of MB-102 (Pig)

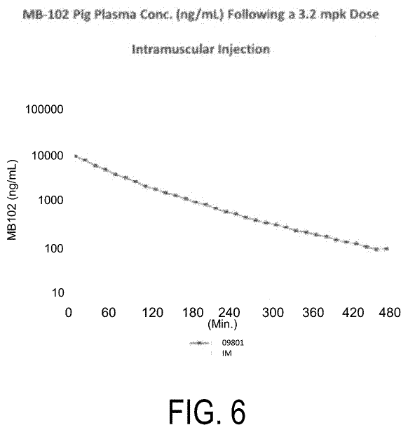

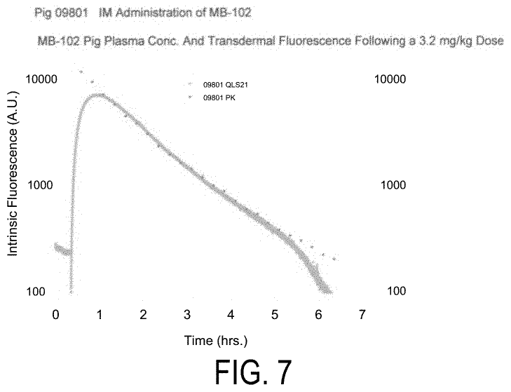

Experimental animal Yorkshire pig, 09801, was subject to Intramuscular (IM) administration of MB-102 using substantially the same procedure as Example 8, but substituting a 2 mL syringe equipped with a single 13 mm×27 g needle. The fluorescence signal was first observed after a few minutes. Blood sampling and processing was as described in Example 8 and the results (Table 1) are plotted in FIG. 6. Fluorescence v. time results are shown in FIG. 7.

In view of the above, it will be seen that the several advantages of the disclosure are achieved and other advantageous results attained. As various changes could be made in the above processes and composites without departing from the scope of the disclosure, it is intended that all matter contained in the above description and shown in the accompanying drawings shall be interpreted as illustrative and not in a limiting sense.

### Example 12—Single Needle Subcutaneous (subQ) Dosing of MB-102 (Pig)

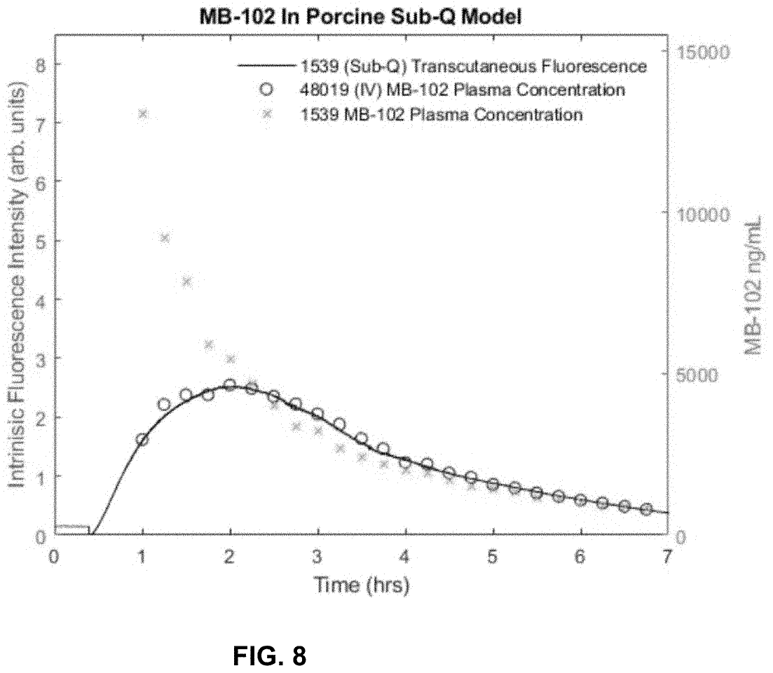

Experimental animal Yorkshire pig, 1539, was subject to subcutaneous (subQ) administration of MB-102 using a single needle in the same protocol as Example 10. The fluorescence signal was first observed after a few minutes. Plasma sampling was conducted as described in Example 8 and the PK results versus time and the companion transdermal fluorescence versus time results are shown in FIG. 8.

What is claimed is:

1. A method for determining glomerular filtration rate (GFR) in a patient in need thereof, said method comprising:
subcutaneously or intramuscularly administering to said patient about 3 mg to about 250 mg of a compound of Formula I or a pharmaceutically acceptable salt thereof as an about 60 mg/ml to an about 300 mg/ml solution, wherein the administration produces a plasma concentration of the compound that is substantially similar to a plasma concentration produced by intravenous administration of an identical amount of the compound;
measuring a concentration of the compound of Formula I in said patient, and
determining the GFR in said patient using the concentration of the compound measured;
wherein Formula I is 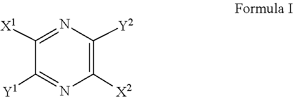
each of X¹ and X² are independently chosen from —CO(AA), —CN, —CO₂R¹, —CONR²R³, —COR⁴, —NO₂, —SOR³⁵, —SO₂R⁸, —SO₂OR⁷ and —PO₃R⁸R⁹;
each Y¹ and Y² are independently chosen from —OR¹⁰, —SR¹¹, —NR¹²R¹³, —N(R¹⁴)COR¹⁵, —CONH(PS); —P(R¹⁶)₂, —P(OR¹⁷)₂; and 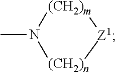
Z¹ is a single bond, —CR¹⁸R¹⁹—, —O—, —NR²⁰—, —NCOR²¹—, —S—, —SO—, and —SO₂—; each R¹ to R²¹ are independently chosen from hydrogen, C₁-C₁₀ alkyl optionally substituted with hydroxyl and carboxylic acid, C₃-C₆ polyhydroxylated alkyl, C₅-C₁₀ aryl, C₅-C₁₀ heteroaryl, C₃-C₅ heterocycloalkyl optionally substituted with C(O), —(CH₂)ₐCO₂H optionally substituted with C₅-C₁₀ heteroaryl, (CH₂)ₐCONR³⁰R³¹, —(CH₂)ₐNHSO₃, —(CH₂)ₐNHSO₃H, —(CH₂)ₐOH, —(CH₂)ₐOPO₃⁼, —(CH₂)ₐOPO₃H₂, —(CH₂)ₐOPO₃H⁻, —(CH₂)ₐOR²², —(CH₂)ₐOSO₃⁻, —(CH₂)ₐOSO₃H, —(CH₂)ₐPO₃⁼, —(CH₂)ₐPO₃H₂, —(CH₂)ₐPO₃H⁻, —(CH₂)ₐSO₃⁻, —(CH₂)ₐSO₃H, —(CH₂)_{d}CO(CH₂CH₂O)_{c}R²³, —(CH₂)_{d}(CH₂CH₂O)_{c}R²⁴, —(CHCO₂H)ₐCO₂H, —CH₂(CHNH₂)ₐCH₂NR²⁵R²⁶, —CH₂(CHOH)ₐCO₂H, —CH₂(CHOH)ₐR²⁷, —CH[(CH₂)_{b}NH₂]ₐCH₂OH, —CH[(CH₂)_{b}NH₂]ₐCO₂H, and —(CH₂)ₐNR²⁸R²⁹; each R²² to R³¹ are independently chosen from hydrogen, C₁-C₁₀alkyl, and C₁-C₅-dicarboxylic acid; R³⁵ is chosen from C₁-C₁₀ alkyl optionally substituted with hydroxyl and carboxylic acid, C₃-C₆ polyhydroxylated alkyl, C₅-C₁₀ aryl, C₅-C₁₀ heteroaryl, C₃-C₅ heterocycloalkyl optionally substituted with C(O), —(CH₂)ₐCO₂H optionally substituted with C₅-C₁₀ heteroaryl, —(CH₂)ₐCONR³⁰R³¹, —(CH₂)ₐNHSO₃⁻, —(CH₂)ₐNHSO₃H, —(CH₂)ₐOH, —(CH₂)ₐOPO₃⁼, —(CH₂)ₐOPO₃H₂, —(CH₂)ₐOPO₃H⁻, —(CH₂)ₐOR²², —(CH₂)ₐOSO₃˜, —(CH₂)ₐOSO₃H, —(CH₂)ₐPO₃˜, —(CH₂)ₐPO₃H₂, —(CH₂)ₐPO₃H⁻, —(CH₂)ₐSO₃⁻, —(CH₂)ₐSO₃H, —(CH₂)_{d}CO(CH₂CH₂O)_{c}R²³, —(CH₂)_{d}(CH₂CH₂O)_{c}R²⁴, —(CHCO₂H)ₐCO₂H, —CH₂(CHNH₂)ₐCH₂NR²⁵R²⁶, —CH₂(CHOH)ₐCO₂H, —CH₂(CHOH)ₐR²⁷, —CH[(CH₂)_{b}NH₂]ₐCH₂OH, —CH[(CH₂)_{b}NH₂]ₐCO₂H, and —(CH₂)ₐNR²⁸R²⁹;
AA is a peptide chain comprising one or more amino acids selected from the group consisting of natural and unnatural amino acids, linked together by peptide or amide bonds and each instance of AA may be the same or different than each other instance;
PS is a sulfated or non-sulfated polysaccharide chain comprising one or more monosaccharide units connected by glycosidic linkages; and
‘a’ is a number from 1 to 10, ‘c’ is a number from 1 to 100, and each of ‘m’ and ‘n’ are independently a number from 1 to 3.

2. The method of claim 1, wherein the concentration of a compound of Formula I in said patient is measured over a measurement time window.

3. The method of claim 1, wherein about 10 mg to about 150 mg of the compound of the compound of Formula I is subcutaneously or intramuscularly administered to a patient.

4. The method of claim 1, wherein the compound of Formula I is subcutaneously or intramuscularly administered to a patient as an about 60 mg/ml to an about 150 mg/ml solution.

5. The method of claim 1, wherein the solution further comprises at least one pharmaceutically acceptable excipient selected from the group consisting of antibacterial agents, antioxidants, buffering agents, osmolarity adjusting agents, pH adjusting agents, preservatives, solvents, stabilizing agents, surfactants, tonicity modifying agents, viscosity adjusting agents, and combinations thereof.

6. The method of claim 5, wherein one of the at least one pharmaceutically acceptable excipient is phosphate buffered saline.

7. The method of claim 1, wherein said patient has a GFR of below 90 as determined in a previous measurement.

8. The method of claim 1, wherein the solution is packaged in a pre-filled syringe.

9. The method of claim 8, wherein the solution is subcutaneously or intramuscularly administered to said patient by an auto-injector.

10. The method of claim 1, wherein a sensor is attached to at least one body site of said patient to detect transdermal fluorescence.

11. The method of claim 10, wherein determining the GFR in said patient using the concentration of the compound measured comprises quantifying and displaying on a mobile computing device the transdermal fluorescence detected by the sensor.

12. The method of claim 1, wherein both X¹ and X² are —CO(AA).

13. The method of claim 12, wherein each instance of AA is a single D-α-amino acid.

14. The method of claim 1, wherein the compound of Formula I is

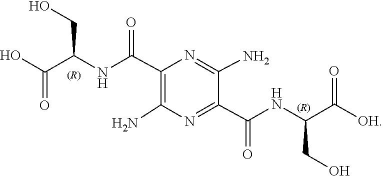

15. The method of claim 1, wherein the pharmaceutically acceptable salt is a cationic or anionic salt.

16. The method of claim 15, wherein the pharmaceutically acceptable salt is a selected from the group consisting of a sodium salt, a choline salt and a meglumine salt.

17. A kit for GFR assessment, the kit comprising:
about 3 mg to about 250 mg of a compound of Formula I or a pharmaceutically acceptable salt thereof as an about 60 mg/ml to an about 300 mg/ml solution,
an injector device configured to subcutaneously or intramuscularly administer the solution into the body of a patient;
a sensor configured to attach to the body of the patient and detect transdermal fluorescence;
a mobile computing device wirelessly communicatively coupled to the sensor and programmed to receive data from the sensor and calculate the GFR of the patient based on the data; and
written instructions describing how to use the components of the kit in order to assess the GFR of the patient;
wherein Formula I is 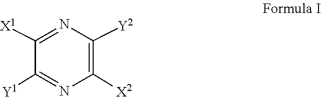
each of X¹ and X² is independently —CO₂R¹, —CONR¹R², —CO(AA) or —CONH(PS);
each of Y¹ and Y² is independently selected from the group consisting of —NR¹R² and 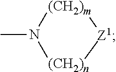
Z¹ is a single bond, —CR¹R²—, —O—, —NR¹—, —NCOR¹—, —S—, —SO—, or —SO₂—;
each of R¹ to R² are independently selected from the group consisting of H, —CH₂(CHOH)ₐH, —CH₂(CHOH)ₐCH₃, —CH₂(CHOH)ₐCO₂H, —(CHCO₂H)ₐCO₂H, —(CH₂CH₂O)_{c}H, —(CH₂CH₂O)_{c}CH₃, —(CH₂)ₐSO₃H, —(CH₂)ₐSO₃⁻, —(CH₂)ₐSO₂H, —(CH₂)ₐSO₂⁻, —(CH₂)ₐNHSO₃H, —(CH₂)ₐNHSO₃⁻, —(CH₂)ₐNHSO₂H, —(CH₂)ₐNHSO₂⁻, —(CH₂)ₐPO₄H₃, —(CH₂)ₐPO₄H₂⁻, —(CH₂)ₐPO₄H²⁻, —(CH₂)ₐPO₄³⁻, —(CH₂)ₐPO₃H₂, —(CH₂)ₐPO₃H⁻, and —(CH₂)ₐPO₃²⁻;
AA is a peptide chain comprising one or more amino acids selected from the group consisting of natural and unnatural amino acids, linked together by peptide or amide bonds and each instance of AA may be the same or different than each other instance;
PS is a sulfated or non-sulfated polysaccharide chain comprising one or more monosaccharide units connected by glycosidic linkages; and
‘a’ is a number from 1 to 10, ‘c’ is a number from 1 to 100, and each of ‘m’ and ‘n’ are independently a number from 1 to 3.
